(19) United States
(12) Patent Application Publication
(10) Pub. No.: US 2018/0243309 A1
(43) Pub. Date: Aug. 30, 2018

(54) TOPICAL ANTIVIRAL COMPOSITIONS

(71) Applicant: Topical Remedy, LLC, Lake Oswego, OR (US)

(72) Inventor: Thomas W. Konowalchuk, Newport, OR (US)

(21) Appl. No.: 15/755,532

(22) PCT Filed: Sep. 1, 2016
(86) PCT No.: PCT/US2016/049982

§ 371 Date: Feb. 26, 2018

Related U.S. Application Data
(60) Provisional application No. 62/214,038, filed on Sep. 3, 2015.

(51) Int. Cl. A61K 31/522; A61K 31/045; A61K 31/19; A61K 9/00; A61P 31/22

(52) U.S. Cl. CPC A61K 31/522; A61K 31/045; A61P 31/22; A61K 9/0014; A61K 31/19

(57) ABSTRACT

In various embodiments compositions that inactivates a virus or inhibit replication of a virus are provided where the compositions comprise a C₁-C₁₀ alcohol; an acid selected from the group consisting of gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid; and a pharmaceutically acceptable carrier; where the pH of said composition is pH 4.5 or lower. In certain embodiments the alcohol comprises one or more alcohols selected from the group consisting of ethanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol.

### CROSS-REFERENCE TO RELATED APPLICATIONS

This application claims priority to and benefit of U.S. Ser. No. 62/214,038, filed on Sep. 3, 2015, which is incorporated herein by reference in its entirety for all purposes.

### STATEMENT OF GOVERNMENTAL SUPPORT

[Not Applicable]

### BACKGROUND

Pathogenic viruses can be classified into two general types with respect to the viral structure, i.e., those that are lipid-enveloped or non-enveloped (naked). Enveloped viruses replicate within the host-cell, recruit viral proteins to the host membrane, and then bud from and utilize the host membrane, essentially, as a vehicle to transport the viral genome to new cellular targets.

Some well-known “enveloped” viruses, include, but are not limited to, Coronavirus (e.g., SARS-CoV), Cytomegalovirus (CMV), Ebola virus, Epstein-Barr virus, Hantavirus, Hepatitis B, Hepatitis C, Herpes simplex (e.g., HS-1, HS-II), Herpes zoster (Varicella-Zoster), Human Immunodeficiency virus (HIV), influenza (including influenza A, influenza B, Avian influenza), Lassa virus (Lassa Fever), Marburg virus, Monkeypox virus, Measles (Morbillivirus), Mumps (Rubulavirus), Parainfluenza virus, Respiratory Syncytial Virus (RSV), and the like.

Approximately 20 to 40% of the adult population experience recurrent outbreaks of Herpes labialis, or cold sores (Spruance and Kriesel (2002) Herpes 9: 64-69). In almost all cases, the disease is due to reactivation of chronic, latent herpes simplex virus type 1 infection from the ganglion of the trigeminal nerve. When the latent infection is activated by any one of a variety of triggers, virions descend through the sensory nerve axons and reinfect the peripheral epithelium. The new Herpes labialis lesion matures within 8 hours after onset (Spruance and Wenerstrom (1984) Oral Surg. Oral Med. Oral Path., 58: 667-671), leaving very little time for successful chemotherapeutic intervention.

A number of products have been marketed as allegedly effective against Herpes infections or other STDs. However, many have proven to be ineffective and the US Food and Drug Administration (FDA) has warned consumers to beware of over a dozen ineffective STD treatments including Medavir, Herpaflor, Viruxo, C-Cure, and Never An Outbreak whose marketing makes unproven claims (see, e.g., www.fda.gov/Drugs/ResourcesForYou/Consumers/BuyingUsingMedicineSafely/MedicationHealthFraud/ucm253405.htm) and warning letters sent by the FDA to the manufacturers, some of which have gone unheeded, stated that various alleged medications were “dangerous to health when used in the manner recommended or suggested in their labeling” (see, e.g., www.fda.gov/ICECI/EnforcementActions/WarningLetters/2011/ucm253516.htm).

The current standard of care for recurrent Herpes labialis involves an oral or topical antiviral given episodically at the onset of each recurrence. A variety of topical over-the-counter preparations are available, but, in most cases, the mechanism of action is ambiguous and very few clinical trials have been performed to define their efficacy. Studies of acyclovir ointment have provided little evidence of efficacy for Herpes labialis in immunocompetent subjects (Spruance et al. (1984) Antimicrob. Agents Chemother. 25: 553-555; Raborn and Grace (2003) J. Can. Dent. Assoc. 5569(8): 498-503). Acyclovir and penciclovir cream-based formulation are both approved for treatment of Herpes labialis, but while both do afford better drug penetration through the skin than the acyclovir ointment, both exhibit relatively limited efficacy with regard to shortening the duration of a herpetic episode (Spruance et al. (2002) Antimicrob. Agents Chemother. 46: 2238-2243; Raborn et al. (2002) J. Am. Dent. Assoc. 133: 303-309). Docosanol 10% cream (Abreva; GlaxoSmithKline) is the most well-studied over-the-counter product, with two randomized trials suggesting some efficacy of treatment. However, because an unrelated placebo was used in these studies, the true level of efficacy cannot be objectively determined (Sailer et al. 92001) Virus Adaption and Treatment 3: 1-6). All these approved topical treatments, as well as Valtrex and Famvir, the approved oral treatments, provide, at best, approximately a one day shortening of the duration of a herpetic episode, and none of them results in a reduction in the percent of the worst outbreaks, the so-called classical lesions. Even the most recently approved new treatment, Xerese, a combination of acyclovir and hydrocortisone, provides less than one day shortening of the herpetic episode, and an only slightly better, 1.5 day, decrease in the time until complete healing of the outbreak. Xerese has been shown to significantly reduce the percent of classical lesion outbreaks, but the increase in the percent of so-called “aborted lesions” with Xerese is only modestly better than the essentially ineffective acyclovir cream (42% for Xerese versus 35% for acyclovir cream) (Id.).

### SUMMARY

In various embodiments compositions are provided herein that have virucidal activity and that are suitable for the treatment and/or prevention of lesions caused by viruses that reside in and/or are transmitted by and/or infect the cells of the dermis or epidermis. Examples of such viruses include, but are not limited to, those of the Herpesviridae family such as Herpes simplex virus, varicella-zoster virus, cytomegalovirus, and Epstein-Barr virus, and viruses of the Poxviridae family such as Molluscum contagiosum. The compositions are also effective against canker sores.

It was a surprising discovery that alcohols and acid that individually are ineffective to inactivate a virus when combined in a single formulation rapidly and effectively inactivate a number of viruses including, inter alia, various herpes viruses. Accordingly in various embodiments, formulations are provided herein that include inter alia an acid and an alcohol.

In various aspects, the invention(s) contemplated herein may include, but need not be limited to, any one or more of the following embodiments:

### Embodiment 1

A composition that inactivates or inhibits replication of a virus, said composition including: a C₁-C₁₀ alcohol; an acid selected from the group consisting of gallic acid, methyl gallate, 3,4-dihydroxy benzoic acid, p-hydroxy benzoic acid, vanillic acid, p-coumaric acid, ferulic acid, syringic acid, salicylic acid, luteic acid, monochloroacetic acid, squaric acid, tartaric acid, retinoic acid, and eudesmic acid; and a pharmaceutically acceptable carrier; where the pH of said composition is pH 4.5 or lower.

### Embodiment 2

A composition that inactivates or inhibits replication of a virus, said composition including: a C₁-C₁₀ alcohol; a beta hydroxy acid or an omega hydroxy acid; and a pharmaceutically acceptable carrier; where the pH of said composition is pH 4.5 or lower.

### Embodiment 3

The composition of embodiment 2, wherein said acid includes a beta hydroxy acid.

### Embodiment 4

The composition of embodiment 3, wherein said acid includes a beta hydroxyl acid selected from the group consisting of salicylic acid, propanoic acid, α-hydroxypropinoic acid, and β-hydroxypropinoic acid.

### Embodiment 5

The composition of embodiment 2, wherein said acid includes an omega hydroxy acid.

### Embodiment 6

The composition of embodiment 5, wherein said acid includes an omega hydroxy acid selected from the group consisting of 16-hydroxy palmitic acid, 18-hydroxy stearic acid, ω-hydroxydotriacontanoic acid, ω-hydroxytetracosanoic acid, ω-hydroxytriacontanoic acid, E)-10-hydroxydec-2-enoic acid, 10-hydroxycapric acid, 12-hydroxylauric acid, 14-hydroxymyristic acid, 22-hydroxydocosanoic acid, 26-hydroxyhexacosanoic acid, 3,12-dihydroxylauric acid, 3,16-dihydroxypalmitic acid, 3,18-dihydroxystearic acid, 5-hydroxypentanoic acid, 6-hydroxyhexanoic acid, and juniperic acid.

### Embodiment 7

The composition according to any one of embodiments 1-6, wherein said composition further includes one or more agents selected from the group consisting of an antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide.

### Embodiment 8

The composition of embodiment 7, wherein said composition includes a long or short chain ceramide.

### Embodiment 9

The composition of embodiment 7, wherein said composition includes a allantoin.

### Embodiment 10

The composition of embodiment 7, wherein said composition includes urea.

### Embodiment 11

The composition of embodiment 7, wherein said composition includes a retinoid.

### Embodiment 12

The composition of embodiment 7, wherein said composition includes an antioxidant phenolic compound selected from the group consisting of phenol, and resorcinol.

### Embodiment 13

The composition of embodiment 7, wherein said composition includes a dicarboxylic acid selected from the group consisting of succinic acid, and tartaric acid.

### Embodiment 14

The composition according to any one of embodiments 1-13, wherein said alcohol includes one or more alcohols selected from the group consisting of methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol.

### Embodiment 15

A composition that inactivates or inhibits replication of a virus, said composition including: a C₁-C₁₀ alcohol; an acid; an additional compound selected from the group consisting of antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide; and a pharmaceutically acceptable carrier; where the pH of said composition is pH 4.5 or lower.

### Embodiment 16

The composition of embodiment 15, wherein said composition includes an antioxidant phenolic compound selected from the group consisting of phenol, and resorcinol.

### Embodiment 17

The composition of embodiment 15, wherein said composition includes a dicarboxylic acid selected from the group consisting of succinic acid, and tartaric acid.

### Embodiment 18

The composition according to any one of embodiments 15-17, wherein said alcohol includes one or more alcohols selected from the group consisting of ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol.

### Embodiment 19

The composition according to any one of embodiments 15-18, wherein said acid includes one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid.

### Embodiment 20

A composition that inactivates or inhibits replication of a virus, said composition including: one or more alcohols selected from the group consisting of butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol; an acid; and a pharmaceutically acceptable carrier; where the pH of said composition is pH 4.5 or lower.

### Embodiment 21

The composition of embodiment 20, wherein said composition further includes one or more agents selected from the group consisting of an antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide.

### Embodiment 22

The composition of embodiment 21, wherein said composition includes an antioxidant phenolic compound selected from the group consisting of phenol, and resorcinol.

### Embodiment 23

The composition of embodiment 21, wherein said composition includes a dicarboxylic acid selected from the group consisting of succinic acid, and tartaric acid.

### Embodiment 24

The composition according to any one of embodiments 20-23, wherein said acid includes one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid.

### Embodiment 25

The composition according to any one of embodiments 1-24, wherein the pH of said composition ranges from pH 2.0 to about pH 4.5.

### Embodiment 26

The composition according to any one of embodiments 1-24, wherein the pH of said composition ranges is about pH 3.5 or lower.

### Embodiment 27

The composition of embodiment 25, wherein the pH of said composition is about pH 3.0 or lower.

### Embodiment 28

The composition of embodiment 25, wherein the pH of said composition is about pH 2.5 or lower.

### Embodiment 29

The composition according to any one of embodiments 1-28, wherein said alcohol ranges from about 0.2% by volume up to about 40% by volume of said composition.

### Embodiment 30

The composition according to any one of embodiments 1-28, wherein said alcohol ranges from about 0.5% by volume up to about 20% by volume of said composition.

### Embodiment 31

The composition according to any one of embodiments 1-28, wherein said alcohol ranges from about 1% by volume up to about 15% by volume of said composition.

### Embodiment 32

The composition according to any one of embodiments 1-31, wherein said composition excludes an amphoteric or pseudoamphoteric compound.

### Embodiment 33

The composition of embodiment 32, wherein said composition excludes an amino acid and/or a peptide.

### Embodiment 34

The composition according to any one of embodiments 32-33, wherein said composition excludes a dipeptide and/or a tripeptide.

### Embodiment 35

The composition according to any one of embodiments 32-34, wherein said composition excludes an imidazoline amphoteric and/or a lecithin amphoteric.

### Embodiment 36

The composition of embodiment 35, wherein said composition excludes one or more compounds selected from the group consisting of cocoamphoglycine, cocoamphoproprionate, cocoamphopropylsulfonate, phosphatidyl ethanolamine, phosphatidyl serine, and sphingomyelin.

### Embodiment 37

The composition according to any one of embodiments 1-36, wherein said composition is formulated as a unit dosage formulation.

### Embodiment 38

The composition according to any one of embodiments 1-37, wherein said composition is formulated as a preparation selected from the group consisting of a fluid, roll-on, spray, tincture, gel, ointment, cream, salve, lotion, lip balm, foam, spray, and aerosol.

### Embodiment 39

A method of inactivating or inhibiting replication of a virus in a mammal, said method including: administering to said mammal a composition according to any one of embodiments 1-38 in an amount sufficient to inhibit, inactivate or kill said virus.

### Embodiment 40

The method of embodiment 39, wherein said administering reduces or inhibits viral-induced lesions in said mammal when said mammal is infected by said virus.

### Embodiment 41

The method of embodiment 39, wherein said virus resides in the dermis or epidermis of a human or animal infected by said virus.

### Embodiment 42

The method according to any one of embodiments 39-41, wherein said composition is applied topically to said mammal.

### Embodiment 43

The method of embodiment 42, wherein said composition is applied to lesions on said mammal.

### Embodiment 44

The method according to any one of embodiments 39-43, wherein said mammal is a human.

### Embodiment 45

The method according to any one of embodiments 39-44, wherein said mammal is infected with said virus or at risk for an infection by said virus.

### Embodiment 46

The method of embodiment 45, wherein said mammal is infected with said virus.

### Embodiment 47

The method according to any one of embodiments 39-46, wherein said virus is a member of the Herpesviridae family.

### Embodiment 48

The method of embodiment 47, wherein said virus is a virus selected from the group consisting of Herpes simplex virus-1 (HSV-1), Herpes simplex virus-2 (HSV-2), Varicella zoster virus (VZV), Epstein-Barr virus (EBV), lymphocryptovirus, Cytomegalovirus (CMV), Roseolovirus, Herpes lymphotropic virus, Pityriasis rosea, and Kaposi's sarcoma-associated herpesvirus (KSHV).

### Embodiment 49

The method of embodiment 47, wherein said virus is Herpes simplex 1.

### Embodiment 50

The method of embodiment 47, wherein said virus is Herpes simplex 2.

### Embodiment 51

The method of embodiment 47, wherein said virus is Varicella zoster virus.

### Embodiment 52

The method according to any one of embodiments 39-42, wherein said virus is a member of the Poxviridae family.

### Embodiment 53

The method of embodiment 52, wherein said virus is a virus selected from the group consisting of smallpox virus (variola), vaccinia virus, cowpox virus, monkeypox virus, orf virus, pseudocowpox, bovine papular stomatitis virus, tanapox virus, Molluscum contagiosum virus (MCV). wherein said mammal is infected with said virus or at risk for an infection by said virus.

### Embodiment 54

The method of embodiment 52, wherein said virus is Molluscum contagiosum.

### Embodiment 55

The method according to any one of embodiments 39-42, wherein said virus is selected from the group consisting of rhinoviruses, adenoviruses, enteroviruses, cornoviruses, respiratory syncytial viruses, influenza viruses and parainfluenza viruses.

It is noted that, in various embodiments, where a glycolic acid is indicated (e.g., in certain of the embodiments listed above), the glycolic acid may be substituted with a glycolate salt (e.g., sodium or potassium salt of glycolates) and where lactic acid is recited, the lactate salt (e.g., sodium or potassium lactate) may be substituted. Similarly in various embodiments, the acid salt of the other recited acids can be substituted therefor.

In certain embodiments any of the compositions described above, and/or below, and/or in the claims exclude an amphoteric or pseudoamphoteric compound. In certain embodiments these compositions exclude an amino acid and/or a peptide and/or a dipeptide and/or a tripeptide, and/or an imidazoline amphoteric, and/or a lecithin amphoteric and/or one or more compounds selected from the group consisting of glycine, cocoamphoglycine, cocoamphoproprionate, cocoamphopropylsulfonate, phosphatidyl ethanolamine, phosphatidyl serine, and sphingomyelin.

In certain embodiments any of the compositions described above, and/or below, and/or in the claims exclude combinations of a C1-C3 alcohol or a C1-C3 diol in combination with any one of glycolic acid, lactic acid, succinic acid, malic acid, citric acid acetic acid, hydrochloric acid.

In certain embodiments any of the compositions described above, and/or below, and/or in the claims exclude any one or more of the following alcohol/acid combinations: ethanol/glycolic acid, methanol/glycolic acid, 1-propanol/glycolic acid, and 2-propanol/glycolic acid, 2,3-butanediol/glycolic acid, 1,2-butanediol/glycolic acid, 1,3-butanediol/glycolic acid, 1,4-butanediol/glycolic acid, ethanol/lactic acid, methanol/lactic acid, 1-propanol/lactic acid, and 2-propanol/lactic acid, 2,3-butanediol/lactic acid, 1,2-butanediol/lactic acid, 1,3-butanediol/lactic acid, 1,4-butanediol/lactic acid, ethanol/succinic acid, methanol/succinic acid, 1-propanol/succinic acid, and 2-propanol/succinic acid, 2,3-butanediol/succinic acid, 1,2-butanediol/succinic acid, 1,3-butanediol/succinic acid, 1,4-butanediol/succinic acid, ethanol/malic acid, methanol/malic acid, 1-propanol/malic acid, and 2-propanol/malic acid, 2,3-butanediol/malic acid, 1,2-butanediol/malic acid, 1,3-butanediol/malic acid, 1,4-butanediol/malic acid, ethanol/citric acid, methanol/citric acid, 1-propanol/citric acid, and 2-propanol/citric acid, 2,3-butanediol/citric acid, 1,2-butanediol/citric acid, 1,3-butanediol/citric acid, 1,4-butanediol/citric acid, ethanol/acetic acid, methanol/acetic acid, 1-propanol/acetic acid, and 2-propanol/acetic acid, 2,3-butanediol/acetic acid, 1,2-butanediol/acetic acid, 1,3-butanediol/acetic acid, 1,4-butanediol/acetic acid, ethanol/hydrochloric acid, methanol/hydrochloric acid, 1-propanol/hydrochloric acid, and 2-propanol/hydrochloric acid, 2,3-butanediol/hydrochloric acid, 1,2-butanediol/hydrochloric acid, 1,3-butanediol/hydrochloric acid, and 1,4-butanediol/hydrochloric acid.

### BRIEF DESCRIPTION OF THE DRAWINGS

[Not Applicable]

### DETAILED DESCRIPTION

In various embodiments compositions are provided herein that have virucidal activity and that are suitable for the treatment and/or prevention of lesions caused by viruses that reside in and/or are transmitted by and/or infect the cells of the dermis or epidermis. Examples of such viruses include, but are not limited to, those of the Herpesviridae family such as Herpes simplex virus, varicella-zoster virus, cytomegalovirus, and Epstein-Barr virus, and viruses of the Poxviridae family such as Molluscum contagiosum. The compositions are also effective against canker sores.

More generally, it is believed the compositions described herein are effective against enveloped viruses including, but not limited to Coronavirus, Cytomegalovirus (CMV), Ebola virus, Epstein-Barr virus, Hantavirus, Hepatitis B, Hepatitis C, Herpes simplex (e.g., HS-1, HS-II), Herpes zoster (Varicella-Zoster), influenza (including influenza A, influenza B, Avian influenza), Lassa virus, Marburg virus, Monkeypox virus, Measles (Morbillivirus), Mumps (Rubulavirus), Parainfluenza virus, Respiratory Syncytial Virus (RSV), and the like.

More specifically, the topical compositions are provided for preventing or reducing canker sores, and/or viral lesions associated with infections from viruses of, for example, the Herpesviridae family or the Poxviridae family, or for shortening the time of healing of such sores or lesions.

In various embodiments the anti-viral compositions contemplated herein comprise an acid and an alcohol, optionally with one more additional active components. In one illustrative embodiment, a composition is provided that is effective to inactivate and/or to inhibit replication of a virus where the composition comprises a C₁-C₁₀ alcohol, or a C₁-C₈ alcohol, or a C₁-C₆ alcohol, or a C₁-C₄ alcohol; and an acid, e.g., an acid selected from the group consisting of gallic acid, methyl gallate, 3,4-dihydroxy benzoic acid, p-hydroxy benzoic acid, vanillic acid, p-coumaric acid, ferulic acid, syringic acid, salicylic acid, luteic acid, monochloroacetic acid, squaric acid, tartaric acid, retinoic acid, and eudesmic acid; and a pharmaceutically acceptable carrier; where the pH of the composition is less than about pH 4.5 or less than about pH 4.0, or less than about pH 3.5, or less than about pH 3.0, or less than about pH 2.5. In certain embodiments the alcohol/acid combination is one selected from the group consisting of ethanol/gallic acid 2-propanol/gallic acid 1-propanol/gallic acid 2,3-butanediol/gallic acid 1,2-butanediol/gallic acid 1,3-butanediol/gallic acid, 1,4-butanediol/gallic acid butyl alcohol/gallic acid n-butanol/gallic acid sec-butanol/gallic acid isobutanol/gallic acid tert-butanol/gallic acid pentanol/gallic acid hexadecan-1-ol/gallic acid ethane-1/gallic acid,-diol/gallic acid propane-1,2-diol/gallic acid propane-1,2,3-triol/gallic acid butane-1,2,3,4-tetraol/gallic acid pentane-1,2,3,4,5-pentol/gallic acid hexane-1,2,3,4,5,6-hexol/gallic acid heptane-1,2,3,4,5,6,7-heptol/gallic acid prop-2-ene-1-ol/gallic acid 3,7-dimethylocta-2,6-dien-1-ol/gallic acid prop-2-in-1-ol/gallic acid cyclohexane-1,2,3,4,5,6-hexol/gallic acid, 2-(2-propyl)-5-methyl-cyclohexane-1-ol/gallic acid, ethanol/methyl gallate 2-propanol/methyl gallate 1-propanol/methyl gallate 2,3-butanediol/methyl gallate 1,2-butanediol/methyl gallate 1,3-butanediol/methyl gallate, 1,4-butanediol/methyl gallate butyl alcohol/methyl gallate n-butanol/methyl gallate sec-butanol/methyl gallate isobutanol/methyl gallate tert-butanol/methyl gallate pentanol/methyl gallate hexadecan-1-ol/methyl gallate ethane-1/methyl gallate,-diol/methyl gallate propane-1,2-diol/methyl gallate propane-1,2,3-triol/methyl gallate butane-1,2,3,4-tetraol/methyl gallate pentane-1,2,3,4,5-pentol/methyl gallate hexane-1,2,3,4,5,6-hexol/methyl gallate heptane-1,2,3,4,5,6,7-heptol/methyl gallate prop-2-ene-1-ol/methyl gallate 3,7-dimethylocta-2,6-dien-1-ol/methyl gallate prop-2-in-1-ol/methyl gallate cyclohexane-1,2,3,4,5,6-hexol/methyl gallate, 2-(2-propyl)-5-methyl-cyclohexane-1-ol/methyl gallate, ethanol/3,4-dihydroxy benzoic acid 2-propanol/3,4-dihydroxy benzoic acid 1-propanol/3,4-dihydroxy benzoic acid 2,3-butanediol/3,4-dihydroxy benzoic acid 1,2-butanediol/3,4-dihydroxy benzoic acid 1,3-butanediol/3,4-dihydroxy benzoic acid, 1,4-butanediol/3,4-dihydroxy benzoic acid butyl alcohol/3,4-dihydroxy benzoic acid n-butanol/3,4-dihydroxy benzoic acid sec-butanol/3,4-dihydroxy benzoic acid isobutanol/3,4-dihydroxy benzoic acid tert-butanol/3,4-dihydroxy benzoic acid pentanol/3,4-dihydroxy benzoic acid hexadecan-1-ol/3,4-dihydroxy benzoic acid ethane-1/3,4-dihydroxy benzoic acid,-diol/3,4-dihydroxy benzoic acid propane-1,2-diol/3,4-dihydroxy benzoic acid propane-1,2,3-triol/3,4-dihydroxy benzoic acid butane-1,2,3,4-tetraol/3,4-dihydroxy benzoic acid pentane-1,2,3,4,5-pentol/3,4-dihydroxy benzoic acid hexane-1,2,3,4,5,6-hexol/3,4-dihydroxy benzoic acid heptane-1,2,3,4,5,6,7-heptol/3,4-dihydroxy benzoic acid prop-2-ene-1-ol/3,4-dihydroxy benzoic acid 3,7-dimethylocta-2,6-dien-1-ol/3,4-dihydroxy benzoic acid prop-2-in-1-ol/3,4-dihydroxy benzoic acid cyclohexane-1,2,3,4,5,6-hexol/3,4-dihydroxy benzoic acid, 2-(2-propyl)-5-methyl-cyclohexane-1-ol/3,4-dihydroxy benzoic acid, ethanol/p-hydroxy benzoic acid 2-propanol/p-hydroxy benzoic acid 1-propanol/p-hydroxy benzoic acid 2,3-butanediol/p-hydroxy benzoic acid 1,2-butanediol/p-hydroxy benzoic acid 1,3-butanediol/p-hydroxy benzoic acid, 1,4-butanediol/p-hydroxy benzoic acid butyl alcohol/p-hydroxy benzoic acid n-butanol/p-hydroxy benzoic acid sec-butanol/p-hydroxy benzoic acid isobutanol/p-hydroxy benzoic acid tert-butanol/p-hydroxy benzoic acid pentanol/p-hydroxy benzoic acid hexadecan-1-ol/p-hydroxy benzoic acid ethane-1/p-hydroxy benzoic acid,-diol/p-hydroxy benzoic acid propane-1,2-diol/p-hydroxy benzoic acid propane-1,2,3-triol/p-hydroxy benzoic acid butane-1,2,3,4-tetraol/p-hydroxy benzoic acid pentane-1,2,3,4,5-pentol/p-hydroxy benzoic acid hexane-1,2,3,4,5,6-hexol/p-hydroxy benzoic acid heptane-1,2,3,4,5,6,7-heptol/p-hydroxy benzoic acid prop-2-ene-1-ol/p-hydroxy benzoic acid 3,7-dimethylocta-2,6-dien-1-ol/p-hydroxy benzoic acid prop-2-in-1-ol/p-hydroxy benzoic acid cyclohexane-1,2,3,4,5,6-hexol/p-hydroxy benzoic acid, 2-(2-propyl)-5-methyl-cyclohexane-1-ol/p-hydroxy benzoic acid, ethanol/vanillic acid 2-propanol/vanillic acid 1-propanol/vanillic acid 2,3-butanediol/vanillic acid 1,2-butanediol/vanillic acid 1,3-butanediol/vanillic acid, 1,4-butanediol/vanillic acid butyl alcohol/vanillic acid n-butanol/vanillic acid sec-butanol/vanillic acid isobutanol/vanillic acid tert-butanol/vanillic acid pentanol/vanillic acid hexadecan-1-ol/vanillic acid ethane-1/vanillic acid,-diol/vanillic acid propane-1,2-diol/vanillic acid propane-1,2,3-triol/vanillic acid butane-1,2,3,4-tetraol/vanillic acid pentane-1,2,3,4,5-pentol/vanillic acid hexane-1,2,3,4,5,6-hexol/vanillic acid heptane-1,2,3,4,5,6,7-heptol/vanillic acid prop-2-ene-1-ol/vanillic acid 3,7-dimethylocta-2,6-dien-1-ol/vanillic acid prop-2-in-1-ol/vanillic acid cyclohexane-1,2,3,4,5,6-hexol/vanillic acid, 2-(2-propyl)-5-methyl-cyclohexane-1-ol/vanillic acid, ethanol/p-coumaric acid 2-propanol/p-coumaric acid 1-propanol/p-coumaric acid 2,3-butanediol/p-coumaric acid 1,2-butanediol/p-coumaric acid 1,3-butanediol/p-coumaric acid, 1,4-butanediol/p-coumaric acid butyl alcohol/p-coumaric acid n-butanol/p-coumaric acid sec-butanol/p-coumaric acid isobutanol/p-coumaric acid tert-butanol/p-coumaric acid pentanol/p-coumaric acid hexadecan-1-ol/p-coumaric acid ethane-1/p-coumaric acid,-diol/p-coumaric acid propane-1,2-diol/p-coumaric acid propane-1,2,3-triol/p-coumaric acid butane-1,2,3,4-tetraol/p-coumaric acid pentane-1,2,3,4,5-pentol/p-coumaric acid hexane-1,2,3,4,5,6-hexol/p-coumaric acid heptane-1,2,3,4,5,6,7-heptol/p-coumaric acid prop-2-ene-1-ol/p-coumaric acid 3,7-dimethylocta-2,6-dien-1-ol/p-coumaric acid prop-2-in-1-ol/p-coumaric acid cyclohexane-1,2,3,4,5,6-hexol/p-coumaric acid, 2-(2-propyl)-5-methyl-cyclohexane-1-ol/p-coumaric acid, ethanol/ferulic acid 2-propanol/ferulic acid 1-propanol/ferulic acid 2,3-butanediol/ferulic acid 1,2-butanediol/ferulic acid 1,3-butanediol/ferulic acid, 1,4-butanediol/ferulic acid butyl alcohol/ferulic acid n-butanol/ferulic acid sec-butanol/ferulic acid isobutanol/ferulic acid tert-butanol/ferulic acid pentanol/ferulic acid hexadecan-1-ol/ferulic acid ethane-1/ferulic acid,-diol/ferulic acid propane-1,2-diol/ferulic acid propane-1,2,3-triol/ferulic acid butane-1,2,3,4-tetraol/ferulic acid pentane-1,2,3,4,5-pentol/ferulic acid hexane-1,2,3,4,5,6-hexol/ferulic acid heptane-1,2,3,4,5,6,7-heptol/ferulic acid prop-2-ene-1-ol/ferulic acid 3,7-dimethylocta-2,6-dien-1-ol/ferulic acid prop-2-in-1-ol/ferulic acid cyclohexane-1,2,3,4,5,6-hexol/ferulic acid, 2-(2-propyl)-5-methyl-cyclohexane-1-ol/ferulic acid, ethanol/syringic acid 2-propanol/syringic acid 1-propanol/syringic acid 2,3-butanediol/syringic acid 1,2-butanediol/syringic acid 1,3-butanediol/syringic acid, 1,4-butanediol/syringic acid butyl alcohol/syringic acid n-butanol/syringic acid sec-butanol/syringic acid isobutanol/syringic acid tert-butanol/syringic acid pentanol/syringic acid hexadecan-1-ol/syringic acid ethane-1/syringic acid,-diol/syringic acid propane-1,2-diol/syringic acid propane-1,2,3-triol/syringic acid butane-1,2,3,4-tetraol/syringic acid pentane-1,2,3,4,5-pentol/syringic acid hexane-1,2,3,4,5,6-hexol/syringic acid heptane-1,2,3,4,5,6,7-heptol/syringic acid prop-2-ene-1-ol/syringic acid 3,7-dimethylocta-2,6-dien-1-ol/syringic acid prop-2-in-1-ol/syringic acid cyclohexane-1,2,3,4,5,6-hexol/syringic acid, 2-(2-propyl)-5-methyl-cyclohexane-1-ol/syringic acid, ethanol/salicylic acid 2-propanol/salicylic acid 1-propanol/salicylic acid 2,3-butanediol/salicylic acid 1,2-butanediol/salicylic acid 1,3-butanediol/salicylic acid, 1,4-butanediol/salicylic acid butyl alcohol/salicylic acid n-butanol/salicylic acid sec-butanol/salicylic acid isobutanol/salicylic acid tert-butanol/salicylic acid pentanol/salicylic acid hexadecan-1-ol/salicylic acid ethane-1/salicylic acid,-diol/salicylic acid propane-1,2-diol/salicylic acid propane-1,2,3-triol/salicylic acid butane-1,2,3,4-tetraol/salicylic acid pentane-1,2,3,4,5-pentol/salicylic acid hexane-1,2,3,4,5,6-hexol/salicylic acid heptane-1,2,3,4,5,6,7-heptol/salicylic acid prop-2-ene-1-ol/salicylic acid 3,7-dimethylocta-2,6-dien-1-ol/salicylic acid prop-2-in-1-ol/salicylic acid cyclohexane-1,2,3,4,5,6-hexol/salicylic acid, 2-(2-propyl)-5-methyl-cyclohexane-1-ol/salicylic acid, ethanol/luteic acid 2-propanol/luteic acid 1-propanol/luteic acid 2,3-butanediol/luteic acid 1,2-butanediol/luteic acid 1,3-butanediol/luteic acid, 1,4-butanediol/luteic acid butyl alcohol/luteic acid n-butanol/luteic acid sec-butanol/luteic acid isobutanol/luteic acid tert-butanol/luteic acid pentanol/luteic acid hexadecan-1-ol/luteic acid ethane-1/luteic acid,-diol/luteic acid propane-1,2-diol/luteic acid propane-1,2,3-triol/luteic acid butane-1,2,3,4-tetraol/luteic acid pentane-1,2,3,4,5-pentol/luteic acid hexane-1,2,3,4,5,6-hexol/luteic acid heptane-1,2,3,4,5,6,7-heptol/luteic acid prop-2-ene-1-ol/luteic acid 3,7-dimethylocta-2,6-dien-1-ol/luteic acid prop-2-in-1-ol/luteic acid cyclohexane-1,2,3,4,5,6-hexol/luteic acid, 2-(2-propyl)-5-methyl-cyclohexane-1-ol/luteic acid, ethanol/eudesmic acid 2-propanol/eudesmic acid 1-propanol/eudesmic acid 2,3-butanediol/eudesmic acid 1,2-butanediol/eudesmic acid 1,3-butanediol/eudesmic acid, 1,4-butanediol/eudesmic acid butyl alcohol/eudesmic acid n-butanol/eudesmic acid sec-butanol/eudesmic acid isobutanol/eudesmic acid tert-butanol/eudesmic acid pentanol/eudesmic acid hexadecan-1-ol/eudesmic acid ethane-1/eudesmic acid,-diol/eudesmic acid propane-1,2-diol/eudesmic acid propane-1,2,3-triol/eudesmic acid butane-1,2,3,4-tetraol/eudesmic acid pentane-1,2,3,4,5-pentol/eudesmic acid hexane-1,2,3,4,5,6-hexol/eudesmic acid heptane-1,2,3,4,5,6,7-heptol/eudesmic acid prop-2-ene-1-ol/eudesmic acid 3,7-dimethylocta-2,6-dien-1-ol/eudesmic acid prop-2-in-1-ol/eudesmic acid cyclohexane-1,2,3,4,5,6-hexol/eudesmic acid, and 2-(2-propyl)-5-methyl-cyclohexane-1-ol/eudesmic acid. In certain embodiments any of these compositions further includes one or more agents selected from the group consisting of an antioxidant phenolic compound, a tannin, a dicarboxylic acids or derivative thereof, mandelic acid, an extract from the plant Prunella vulgaris, and vitamin C. In certain embodiments any of these compositions further comprises an antioxidant phenolic compound selected from the group consisting of phenol, and resorcinol. In certain embodiments any of these compositions further comprises a dicarboxylic acid selected from the group consisting of succinic acid, and tartaric acid.

In another illustrative, but non-limiting embodiment, a composition is provided that is effective to inactivate and/or to inhibit replication of a virus where the composition comprises a comprises a C₁-C₁₀ alcohol, or a C₁-C₈ alcohol, or a C₁-C₆ alcohol, or a C₁-C₄ alcohol; a beta hydroxy acid or an omega hydroxy acid; and a pharmaceutically acceptable carrier; where the pH of said composition is pH 4.5 or lower. In certain embodiments the acid comprises a beta hydroxy acid (e.g., salicylic acid, propanoic acid, α-hydroxypropinoic acid, β-hydroxypropinoic acid, etc.). In certain embodiments the acid comprises an omega hydroxy acid (e.g., 16-hydroxy palmitic acid, 18-hydroxy stearic acid, ω-hydroxydotriacontanoic acid, ω-hydroxytetracosanoic acid, ω-hydroxytriacontanoic acid, E)-10-hydroxydec-2-enoic acid, 10-hydroxycapric acid, 12-hydroxylauric acid, 14-hydroxymyristic acid, 22-hydroxydocosanoic acid, 26-hydroxyhexacosanoic acid, 3,12-dihydroxylauric acid, 3,16-dihydroxypalmitic acid, 3,18-dihydroxystearic acid, 5-hydroxypentanoic acid, 6-hydroxyhexanoic acid, juniperic acid, etc.).

In still another illustrative embodiment a composition is provided that inactivates or inhibits replication of a virus, where the composition comprises a C₁-C₁₀ alcohol, or a C₁-C₈ alcohol, or a C₁-C₆ alcohol, or a C₁-C₄ alcohol; an acid; an additional compound selected from the group consisting of an antioxidant phenolic compound, a tannin, a dicarboxylic acid or derivative thereof, mandelic acid, an extract from the plant Prunella vulgaris, and vitamin C; and a pharmaceutically acceptable carrier; where the pH of the composition is less than about pH 4.5. In certain embodiments this composition comprises an antioxidant phenolic compound selected from the group consisting of phenol, and resorcinol. In certain embodiments this composition comprises a dicarboxylic acid selected from the group consisting of succinic acid, and tartaric acid. In certain embodiments this composition comprises one or more alcohols selected from the group consisting of ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), Pentanol, Hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol. In certain embodiments this composition comprises one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid. In certain embodiments the alcohol/acid combination comprising this composition is one selected from the group consisting of ethanol/glycolic acid, ethanol/lactic acid, ethanol/succinic acid, ethanol/malic acid, ethanol/citric acid, ethanol/acetic acid, ethanol/formic acid, ethanol/oxalic acid, ethanol/uric acid, ethanol/hydrochloric acid, ethanol/nitric acid, ethanol/phosphoric acid, ethanol/sulphuric acid, ethanol/boric acid, ethanol/hydrobromic acid, ethanol/hydroiodic acid, ethanol/hydrosulfuric acid, ethanol/perchloric acid, ethanol/gallic acid, ethanol/methyl gallate, ethanol/3, ethanol/4-dihydroxy benzoic acid, ethanol/p-hydroxy benzoic acid, ethanol/vanillic acid, ethanol/p-coumaric acid, ethanol/ferulic acid, ethanol/syringic acid, ethanol/salicylic acid, ethanol/luteic acid, ethanol/eudesmic acid, 2-propanol/glycolic acid, 2-propanol/lactic acid, 2-propanol/succinic acid, 2-propanol/malic acid, 2-propanol/citric acid, 2-propanol/acetic acid, 2-propanol/formic acid, 2-propanol/oxalic acid, 2-propanol/uric acid, 2-propanol/hydrochloric acid, 2-propanol/nitric acid, 2-propanol/phosphoric acid, 2-propanol/sulphuric acid, 2-propanol/boric acid, 2-propanol/hydrobromic acid, 2-propanol/hydroiodic acid, 2-propanol/hydrosulfuric acid, 2-propanol/perchloric acid, 2-propanol/gallic acid, 2-propanol/methyl gallate, 2-propanol/3,2-propanol/4-dihydroxy benzoic acid, 2-propanol/p-hydroxy benzoic acid, 2-propanol/vanillic acid, 2-propanol/p-coumaric acid, 2-propanol/ferulic acid, 2-propanol/syringic acid, 2-propanol/salicylic acid, 2-propanol/luteic acid, 2-propanol/eudesmic acid, 1-propanol/glycolic acid, 1-propanol/lactic acid, 1-propanol/succinic acid, 1-propanol/malic acid, 1-propanol/citric acid, 1-propanol/acetic acid, 1-propanol/formic acid, 1-propanol/oxalic acid, 1-propanol/uric acid, 1-propanol/hydrochloric acid, 1-propanol/nitric acid, 1-propanol/phosphoric acid, 1-propanol/sulphuric acid, 1-propanol/boric acid, 1-propanol/hydrobromic acid, 1-propanol/hydroiodic acid, 1-propanol/hydrosulfuric acid, 1-propanol/perchloric acid, 1-propanol/gallic acid, 1-propanol/methyl gallate, 1-propanol/3,1-propanol/4-dihydroxy benzoic acid, 1-propanol/p-hydroxy benzoic acid, 1-propanol/vanillic acid, 1-propanol/p-coumaric acid, 1-propanol/ferulic acid, 1-propanol/syringic acid, 1-propanol/salicylic acid, 1-propanol/luteic acid, 1-propanol/eudesmic acid, 2,3-butanediol/glycolic acid, 2,3-butanediol/lactic acid, 2,3-butanediol/succinic acid, 2,3-butanediol/malic acid, 2,3-butanediol/citric acid, 2,3-butanediol/acetic acid, 2,3-butanediol/formic acid, 2,3-butanediol/oxalic acid, 2,3-butanediol/uric acid, 2,3-butanediol/hydrochloric acid, 2,3-butanediol/nitric acid, 2,3-butanediol/phosphoric acid, 2,3-butanediol/sulphuric acid, 2,3-butanediol/boric acid, 2,3-butanediol/hydrobromic acid, 2,3-butanediol/hydroiodic acid, 2,3-butanediol/hydrosulfuric acid, 2,3-butanediol/perchloric acid, 2,3-butanediol/gallic acid, 2,3-butanediol/methyl gallate, 2,3-butanediol/3,2,3-butanediol/4-dihydroxy benzoic acid, 2,3-butanediol/p-hydroxy benzoic acid, 2,3-butanediol/vanillic acid, 2,3-butanediol/p-coumaric acid, 2,3-butanediol/ferulic acid, 2,3-butanediol/syringic acid, 2,3-butanediol/salicylic acid, 2,3-butanediol/luteic acid, 2,3-butanediol/eudesmic acid, 1,2-butanediol/glycolic acid, 1,2-butanediol/lactic acid, 1,2-butanediol/succinic acid, 1,2-butanediol/malic acid, 1,2-butanediol/citric acid, 1,2-butanediol/acetic acid, 1,2-butanediol/formic acid, 1,2-butanediol/oxalic acid, 1,2-butanediol/uric acid, 1,2-butanediol/hydrochloric acid, 1,2-butanediol/nitric acid, 1,2-butanediol/phosphoric acid, 1,2-butanediol/sulphuric acid, 1,2-butanediol/boric acid, 1,2-butanediol/hydrobromic acid, 1,2-butanediol/hydroiodic acid, 1,2-butanediol/hydrosulfuric acid, 1,2-butanediol/perchloric acid, 1,2-butanediol/gallic acid, 1,2-butanediol/methyl gallate, 1,2-butanediol/3,1,2-butanediol/4-dihydroxy benzoic acid, 1,2-butanediol/p-hydroxy benzoic acid, 1,2-butanediol/vanillic acid, 1,2-butanediol/p-coumaric acid, 1,2-butanediol/ferulic acid, 1,2-butanediol/syringic acid, 1,2-butanediol/salicylic acid, 1,2-butanediol/luteic acid, 1,2-butanediol/eudesmic acid, 1,3-butanediol/glycolic acid, 1,3-butanediol/lactic acid, 1,3-butanediol/succinic acid, 1,3-butanediol/malic acid, 1,3-butanediol/citric acid, 1,3-butanediol/acetic acid, 1,3-butanediol/formic acid, 1,3-butanediol/oxalic acid, 1,3-butanediol/uric acid, 1,3-butanediol/hydrochloric acid, 1,3-butanediol/nitric acid, 1,3-butanediol/phosphoric acid, 1,3-butanediol/sulphuric acid, 1,3-butanediol/boric acid, 1,3-butanediol/hydrobromic acid, 1,3-butanediol/hydroiodic acid, 1,3-butanediol/hydrosulfuric acid, 1,3-butanediol/perchloric acid, 1,3-butanediol/gallic acid, 1,3-butanediol/methyl gallate, 1,3-butanediol/3,1,3-butanediol/4-dihydroxy benzoic acid, 1,3-butanediol/p-hydroxy benzoic acid, 1,3-butanediol/vanillic acid, 1,3-butanediol/p-coumaric acid, 1,3-butanediol/ferulic acid, 1,3-butanediol/syringic acid, 1,3-butanediol/salicylic acid, 1,3-butanediol/luteic acid, 1,3-butanediol/eudesmic acid, 1,4-butanediol/glycolic acid, 1,4-butanediol/lactic acid, 1,4-butanediol/succinic acid, 1,4-butanediol/malic acid, 1,4-butanediol/citric acid, 1,4-butanediol/acetic acid, 1,4-butanediol/formic acid, 1,4-butanediol/oxalic acid, 1,4-butanediol/uric acid, 1,4-butanediol/hydrochloric acid, 1,4-butanediol/nitric acid, 1,4-butanediol/phosphoric acid, 1,4-butanediol/sulphuric acid, 1,4-butanediol/boric acid, 1,4-butanediol/hydrobromic acid, 1,4-butanediol/hydroiodic acid, 1,4-butanediol/hydrosulfuric acid, 1,4-butanediol/perchloric acid, 1,4-butanediol/gallic acid, 1,4-butanediol/methyl gallate, 1,4-butanediol/3,1,4-butanediol/4-dihydroxy benzoic acid, 1,4-butanediol/p-hydroxy benzoic acid, 1,4-butanediol/vanillic acid, 1,4-butanediol/p-coumaric acid, 1,4-butanediol/ferulic acid, 1,4-butanediol/syringic acid, 1,4-butanediol/salicylic acid, 1,4-butanediol/luteic acid, 1,4-butanediol/eudesmic acid, butyl alcohol/glycolic acid, butyl alcohol/lactic acid, butyl alcohol/succinic acid, butyl alcohol/malic acid, butyl alcohol/citric acid, butyl alcohol/acetic acid, butyl alcohol/formic acid, butyl alcohol/oxalic acid, butyl alcohol/uric acid, butyl alcohol/hydrochloric acid, butyl alcohol/nitric acid, butyl alcohol/phosphoric acid, butyl alcohol/sulphuric acid, butyl alcohol/boric acid, butyl alcohol/hydrobromic acid, butyl alcohol/hydroiodic acid, butyl alcohol/hydrosulfuric acid, butyl alcohol/perchloric acid, butyl alcohol/gallic acid, butyl alcohol/methyl gallate, butyl alcohol/3, butyl alcohol/4-dihydroxy benzoic acid, butyl alcohol/p-hydroxy benzoic acid, butyl alcohol/vanillic acid, butyl alcohol/p-coumaric acid, butyl alcohol/ferulic acid, butyl alcohol/syringic acid, butyl alcohol/salicylic acid, butyl alcohol/luteic acid, butyl alcohol/eudesmic acid, n-butanol/glycolic acid, n-butanol/lactic acid, n-butanol/succinic acid, n-butanol/malic acid, n-butanol/citric acid, n-butanol/acetic acid, n-butanol/formic acid, n-butanol/oxalic acid, n-butanol/uric acid, n-butanol/hydrochloric acid, n-butanol/nitric acid, n-butanol/phosphoric acid, n-butanol/sulphuric acid, n-butanol/boric acid, n-butanol/hydrobromic acid, n-butanol/hydroiodic acid, n-butanol/hydrosulfuric acid, n-butanol/perchloric acid, n-butanol/gallic acid, n-butanol/methyl gallate, n-butanol/3, n-butanol/4-dihydroxy benzoic acid, n-butanol/p-hydroxy benzoic acid, n-butanol/vanillic acid, n-butanol/p-coumaric acid, n-butanol/ferulic acid, n-butanol/syringic acid, n-butanol/salicylic acid, n-butanol/luteic acid, n-butanol/eudesmic acid, sec-butanol/glycolic acid, sec-butanol/lactic acid, sec-butanol/succinic acid, sec-butanol/malic acid, sec-butanol/citric acid, sec-butanol/acetic acid, sec-butanol/formic acid, sec-butanol/oxalic acid, sec-butanol/uric acid, sec-butanol/hydrochloric acid, sec-butanol/nitric acid, sec-butanol/phosphoric acid, sec-butanol/sulphuric acid, sec-butanol/boric acid, sec-butanol/hydrobromic acid, sec-butanol/hydroiodic acid, sec-butanol/hydrosulfuric acid, sec-butanol/perchloric acid, sec-butanol/gallic acid, sec-butanol/methyl gallate, sec-butanol/3, sec-butanol/4-dihydroxy benzoic acid, sec-butanol/p-hydroxy benzoic acid, sec-butanol/vanillic acid, sec-butanol/p-coumaric acid, sec-butanol/ferulic acid, sec-butanol/syringic acid, sec-butanol/salicylic acid, sec-butanol/luteic acid, sec-butanol/eudesmic acid, isobutanol/glycolic acid, isobutanol/lactic acid, isobutanol/succinic acid, isobutanol/malic acid, isobutanol/citric acid, isobutanol/acetic acid, isobutanol/formic acid, isobutanol/oxalic acid, isobutanol/uric acid, isobutanol/hydrochloric acid, isobutanol/nitric acid, isobutanol/phosphoric acid, isobutanol/sulphuric acid, isobutanol/boric acid, isobutanol/hydrobromic acid, isobutanol/hydroiodic acid, isobutanol/hydrosulfuric acid, isobutanol/perchloric acid, isobutanol/gallic acid, isobutanol/methyl gallate, isobutanol/3, isobutanol/4-dihydroxy benzoic acid, isobutanol/p-hydroxy benzoic acid, isobutanol/vanillic acid, isobutanol/p-coumaric acid, isobutanol/ferulic acid, isobutanol/syringic acid, isobutanol/salicylic acid, isobutanol/luteic acid, isobutanol/eudesmic acid, tert-butanol/glycolic acid, tert-butanol/lactic acid, tert-butanol/succinic acid, tert-butanol/malic acid, tert-butanol/citric acid, tert-butanol/acetic acid, tert-butanol/formic acid, tert-butanol/oxalic acid, tert-butanol/uric acid, tert-butanol/hydrochloric acid, tert-butanol/nitric acid, tert-butanol/phosphoric acid, tert-butanol/sulphuric acid, tert-butanol/boric acid, tert-butanol/hydrobromic acid, tert-butanol/hydroiodic acid, tert-butanol/hydrosulfuric acid, tert-butanol/perchloric acid, tert-butanol/gallic acid, tert-butanol/methyl gallate, tert-butanol/3, tert-butanol/4-dihydroxy benzoic acid, tert-butanol/p-hydroxy benzoic acid, tert-butanol/vanillic acid, tert-butanol/p-coumaric acid, tert-butanol/ferulic acid, tert-butanol/syringic acid, tert-butanol/salicylic acid, tert-butanol/luteic acid, tert-butanol/eudesmic acid, pentanol/glycolic acid, pentanol/lactic acid, pentanol/succinic acid, pentanol/malic acid, pentanol/citric acid, pentanol/acetic acid, pentanol/formic acid, pentanol/oxalic acid, pentanol/uric acid, pentanol/hydrochloric acid, pentanol/nitric acid, pentanol/phosphoric acid, pentanol/sulphuric acid, pentanol/boric acid, pentanol/hydrobromic acid, pentanol/hydroiodic acid, pentanol/hydrosulfuric acid, pentanol/perchloric acid, pentanol/gallic acid, pentanol/methyl gallate, pentanol/3, pentanol/4-dihydroxy benzoic acid, pentanol/p-hydroxy benzoic acid, pentanol/vanillic acid, pentanol/p-coumaric acid, pentanol/ferulic acid, pentanol/syringic acid, pentanol/salicylic acid, pentanol/luteic acid, pentanol/eudesmic acid, hexadecan-1-ol/glycolic acid, hexadecan-1-ol/lactic acid, hexadecan-1-ol/succinic acid, hexadecan-1-ol/malic acid, hexadecan-1-ol/citric acid, hexadecan-1-ol/acetic acid, hexadecan-1-ol/formic acid, hexadecan-1-ol/oxalic acid, hexadecan-1-ol/uric acid, hexadecan-1-ol/hydrochloric acid, hexadecan-1-ol/nitric acid, hexadecan-1-ol/phosphoric acid, hexadecan-1-ol/sulphuric acid, hexadecan-1-ol/boric acid, hexadecan-1-ol/hydrobromic acid, hexadecan-1-ol/hydroiodic acid, hexadecan-1-ol/hydrosulfuric acid, hexadecan-1-ol/perchloric acid, hexadecan-1-ol/gallic acid, hexadecan-1-ol/methyl gallate, hexadecan-1-ol/3, hexadecan-1-ol/4-dihydroxy benzoic acid, hexadecan-1-ol/p-hydroxy benzoic acid, hexadecan-1-ol/vanillic acid, hexadecan-1-ol/p-coumaric acid, hexadecan-1-ol/ferulic acid, hexadecan-1-ol/syringic acid, hexadecan-1-ol/salicylic acid, hexadecan-1-ol/luteic acid, hexadecan-1-ol/eudesmic acid, ethane-1,2-diol/glycolic acid, ethane-1,2-diol/lactic acid, ethane-1,2-diol/succinic acid, ethane-1,2-diol/malic acid, ethane-1,2-diol/citric acid, ethane-1,2-diol/acetic acid, ethane-1,2-diol/formic acid, ethane-1,2-diol/oxalic acid, ethane-1,2-diol/uric acid, ethane-1,2-diol/hydrochloric acid, ethane-1,2-diol/nitric acid, ethane-1,2-diol/phosphoric acid, ethane-1,2-diol/sulphuric acid, ethane-1,2-diol/boric acid, ethane-1,2-diol/hydrobromic acid, ethane-1,2-diol/hydroiodic acid, ethane-1,2-diol/hydrosulfuric acid, ethane-1,2-diol/perchloric acid, ethane-1,2-diol/gallic acid, ethane-1,2-diol/methyl gallate, ethane-1,2-diol/3, ethane-1,2-diol/4-dihydroxy benzoic acid, ethane-1,2-diol/p-hydroxy benzoic acid, ethane-1,2-diol/vanillic acid, ethane-1,2-diol/p-coumaric acid, ethane-1,2-diol/ferulic acid, ethane-1,2-diol/syringic acid, ethane-1,2-diol/salicylic acid, ethane-1,2-diol/luteic acid, ethane-1,2-diol/eudesmic acid, propane-1,2-diol/glycolic acid, propane-1,2-diol/lactic acid, propane-1,2-diol/succinic acid, propane-1,2-diol/malic acid, propane-1,2-diol/citric acid, propane-1,2-diol/acetic acid, propane-1,2-diol/formic acid, propane-1,2-diol/oxalic acid, propane-1,2-diol/uric acid, propane-1,2-diol/hydrochloric acid, propane-1,2-diol/nitric acid, propane-1,2-diol/phosphoric acid, propane-1,2-diol/sulphuric acid, propane-1,2-diol/boric acid, propane-1,2-diol/hydrobromic acid, propane-1,2-diol/hydroiodic acid, propane-1,2-diol/hydrosulfuric acid, propane-1,2-diol/perchloric acid, propane-1,2-diol/gallic acid, propane-1,2-diol/methyl gallate, propane-1,2-diol/3, propane-1,2-diol/4-dihydroxy benzoic acid, propane-1,2-diol/p-hydroxy benzoic acid, propane-1,2-diol/vanillic acid, propane-1,2-diol/p-coumaric acid, propane-1,2-diol/ferulic acid, propane-1,2-diol/syringic acid, propane-1,2-diol/salicylic acid, propane-1,2-diol/luteic acid, propane-1,2-diol/eudesmic acid, propane-1,2,3-triol/glycolic acid, propane-1,2,3-triol/lactic acid, propane-1,2,3-triol/succinic acid, propane-1,2,3-triol/malic acid, propane-1,2,3-triol/citric acid, propane-1,2,3-triol/acetic acid, propane-1,2,3-triol/formic acid, propane-1,2,3-triol/oxalic acid, propane-1,2,3-triol/uric acid, propane-1,2,3-triol/hydrochloric acid, propane-1,2,3-triol/nitric acid, propane-1,2,3-triol/phosphoric acid, propane-1,2,3-triol/sulphuric acid, propane-1,2,3-triol/boric acid, propane-1,2,3-triol/hydrobromic acid, propane-1,2,3-triol/hydroiodic acid, propane-1,2,3-triol/hydrosulfuric acid, propane-1,2,3-triol/perchloric acid, propane-1,2,3-triol/gallic acid, propane-1,2,3-triol/methyl gallate, propane-1,2,3-triol/3, propane-1,2,3-triol/4-dihydroxy benzoic acid, propane-1,2,3-triol/p-hydroxy benzoic acid, propane-1,2,3-triol/vanillic acid, propane-1,2,3-triol/p-coumaric acid, propane-1,2,3-triol/ferulic acid, propane-1,2,3-triol/syringic acid, propane-1,2,3-triol/salicylic acid, propane-1,2,3-triol/luteic acid, propane-1,2,3-triol/eudesmic acid, butane-1,2,3,4-tetraol/glycolic acid, butane-1,2,3,4-tetraol/lactic acid, butane-1,2,3,4-tetraol/succinic acid, butane-1,2,3,4-tetraol/malic acid, butane-1,2,3,4-tetraol/citric acid, butane-1,2,3,4-tetraol/acetic acid, butane-1,2,3,4-tetraol/formic acid, butane-1,2,3,4-tetraol/oxalic acid, butane-1,2,3,4-tetraol/uric acid, butane-1,2,3,4-tetraol/hydrochloric acid, butane-1,2,3,4-tetraol/nitric acid, butane-1,2,3,4-tetraol/phosphoric acid, butane-1,2,3,4-tetraol/sulphuric acid, butane-1,2,3,4-tetraol/boric acid, butane-1,2,3,4-tetraol/hydrobromic acid, butane-1,2,3,4-tetraol/hydroiodic acid, butane-1,2,3,4-tetraol/hydrosulfuric acid, butane-1,2,3,4-tetraol/perchloric acid, butane-1,2,3,4-tetraol/gallic acid, butane-1,2,3,4-tetraol/methyl gallate, butane-1,2,3,4-tetraol/3, butane-1,2,3,4-tetraol/4-dihydroxy benzoic acid, butane-1,2,3,4-tetraol/p-hydroxy benzoic acid, butane-1,2,3,4-tetraol/vanillic acid, butane-1,2,3,4-tetraol/p-coumaric acid, butane-1,2,3,4-tetraol/ferulic acid, butane-1,2,3,4-tetraol/syringic acid, butane-1,2,3,4-tetraol/salicylic acid, butane-1,2,3,4-tetraol/luteic acid, butane-1,2,3,4-tetraol/eudesmic acid, pentane-1,2,3,4,5-pentol/glycolic acid, pentane-1,2,3,4,5-pentol/lactic acid, pentane-1,2,3,4,5-pentol/succinic acid, pentane-1,2,3,4,5-pentol/malic acid, pentane-1,2,3,4,5-pentol/citric acid, pentane-1,2,3,4,5-pentol/acetic acid, pentane-1,2,3,4,5-pentol/formic acid, pentane-1,2,3,4,5-pentol/oxalic acid, pentane-1,2,3,4,5-pentol/uric acid, pentane-1,2,3,4,5-pentol/hydrochloric acid, pentane-1,2,3,4,5-pentol/nitric acid, pentane-1,2,3,4,5-pentol/phosphoric acid, pentane-1,2,3,4,5-pentol/sulphuric acid, pentane-1,2,3,4,5-pentol/boric acid, pentane-1,2,3,4,5-pentol/hydrobromic acid, pentane-1,2,3,4,5-pentol/hydroiodic acid, pentane-1,2,3,4,5-pentol/hydrosulfuric acid, pentane-1,2,3,4,5-pentol/perchloric acid, pentane-1,2,3,4,5-pentol/gallic acid, pentane-1,2,3,4,5-pentol/methyl gallate, pentane-1,2,3,4,5-pentol/3, pentane-1,2,3,4,5-pentol/4-dihydroxy benzoic acid, pentane-1,2,3,4,5-pentol/p-hydroxy benzoic acid, pentane-1,2,3,4,5-pentol/vanillic acid, pentane-1,2,3,4,5-pentol/p-coumaric acid, pentane-1,2,3,4,5-pentol/ferulic acid, pentane-1,2,3,4,5-pentol/syringic acid, pentane-1,2,3,4,5-pentol/salicylic acid, pentane-1,2,3,4,5-pentol/luteic acid, pentane-1,2,3,4,5-pentol/eudesmic acid, hexane-1,2,3,4,5,6-hexol/glycolic acid, hexane-1,2,3,4,5,6-hexol/lactic acid, hexane-1,2,3,4,5,6-hexol/succinic acid, hexane-1,2,3,4,5,6-hexol/malic acid, hexane-1,2,3,4,5,6-hexol/citric acid, hexane-1,2,3,4,5,6-hexol/acetic acid, hexane-1,2,3,4,5,6-hexol/formic acid, hexane-1,2,3,4,5,6-hexol/oxalic acid, hexane-1,2,3,4,5,6-hexol/uric acid, hexane-1,2,3,4,5,6-hexol/hydrochloric acid, hexane-1,2,3,4,5,6-hexol/nitric acid, hexane-1,2,3,4,5,6-hexol/phosphoric acid, hexane-1,2,3,4,5,6-hexol/sulphuric acid, hexane-1,2,3,4,5,6-hexol/boric acid, hexane-1,2,3,4,5,6-hexol/hydrobromic acid, hexane-1,2,3,4,5,6-hexol/hydroiodic acid, hexane-1,2,3,4,5,6-hexol/hydrosulfuric acid, hexane-1,2,3,4,5,6-hexol/perchloric acid, hexane-1,2,3,4,5,6-hexol/gallic acid, hexane-1,2,3,4,5,6-hexol/methyl gallate, hexane-1,2,3,4,5,6-hexol/3, hexane-1,2,3,4,5,6-hexol/4-dihydroxy benzoic acid, hexane-1,2,3,4,5,6-hexol/p-hydroxy benzoic acid, hexane-1,2,3,4,5,6-hexol/vanillic acid, hexane-1,2,3,4,5,6-hexol/p-coumaric acid, hexane-1,2,3,4,5,6-hexol/ferulic acid, hexane-1,2,3,4,5,6-hexol/syringic acid, hexane-1,2,3,4,5,6-hexol/salicylic acid, hexane-1,2,3,4,5,6-hexol/luteic acid, hexane-1,2,3,4,5,6-hexol/eudesmic acid, heptane-1,2,3,4,5,6,7-heptol/glycolic acid, heptane-1,2,3,4,5,6,7-heptol/lactic acid, heptane-1,2,3,4,5,6,7-heptol/succinic acid, heptane-1,2,3,4,5,6,7-heptol/malic acid, heptane-1,2,3,4,5,6,7-heptol/citric acid, heptane-1,2,3,4,5,6,7-heptol/acetic acid, heptane-1,2,3,4,5,6,7-heptol/formic acid, heptane-1,2,3,4,5,6,7-heptol/oxalic acid, heptane-1,2,3,4,5,6,7-heptol/uric acid, heptane-1,2,3,4,5,6,7-heptol/hydrochloric acid, heptane-1,2,3,4,5,6,7-heptol/nitric acid, heptane-1,2,3,4,5,6,7-heptol/phosphoric acid, heptane-1,2,3,4,5,6,7-heptol/sulphuric acid, heptane-1,2,3,4,5,6,7-heptol/boric acid, heptane-1,2,3,4,5,6,7-heptol/hydrobromic acid, heptane-1,2,3,4,5,6,7-heptol/hydroiodic acid, heptane-1,2,3,4,5,6,7-heptol/hydrosulfuric acid, heptane-1,2,3,4,5,6,7-heptol/perchloric acid, heptane-1,2,3,4,5,6,7-heptol/gallic acid, heptane-1,2,3,4,5,6,7-heptol/methyl gallate, heptane-1,2,3,4,5,6,7-heptol/3, heptane-1,2,3,4,5,6,7-heptol/4-dihydroxy benzoic acid, heptane-1,2,3,4,5,6,7-heptol/p-hydroxy benzoic acid, heptane-1,2,3,4,5,6,7-heptol/vanillic acid, heptane-1,2,3,4,5,6,7-heptol/p-coumaric acid, heptane-1,2,3,4,5,6,7-heptol/ferulic acid, heptane-1,2,3,4,5,6,7-heptol/syringic acid, heptane-1,2,3,4,5,6,7-heptol/salicylic acid, heptane-1,2,3,4,5,6,7-heptol/luteic acid, heptane-1,2,3,4,5,6,7-heptol/eudesmic acid, prop-2-ene-1-ol/glycolic acid, prop-2-ene-1-ol/lactic acid, prop-2-ene-1-ol/succinic acid, prop-2-ene-1-ol/malic acid, prop-2-ene-1-ol/citric acid, prop-2-ene-1-ol/acetic acid, prop-2-ene-1-ol/formic acid, prop-2-ene-1-ol/oxalic acid, prop-2-ene-1-ol/uric acid, prop-2-ene-1-ol/hydrochloric acid, prop-2-ene-1-ol/nitric acid, prop-2-ene-1-ol/phosphoric acid, prop-2-ene-1-ol/sulphuric acid, prop-2-ene-1-ol/boric acid, prop-2-ene-1-ol/hydrobromic acid, prop-2-ene-1-ol/hydroiodic acid, prop-2-ene-1-ol/hydrosulfuric acid, prop-2-ene-1-ol/perchloric acid, prop-2-ene-1-ol/gallic acid, prop-2-ene-1-ol/methyl gallate, prop-2-ene-1-ol/3, prop-2-ene-1-ol/4-dihydroxy benzoic acid, prop-2-ene-1-ol/p-hydroxy benzoic acid, prop-2-ene-1-ol/vanillic acid, prop-2-ene-1-ol/p-coumaric acid, prop-2-ene-1-ol/ferulic acid, prop-2-ene-1-ol/syringic acid, prop-2-ene-1-ol/salicylic acid, prop-2-ene-1-ol/luteic acid, prop-2-ene-1-ol/eudesmic acid, 3,7-dimethylocta-2,6-dien-1-ol/glycolic acid, 3,7-dimethylocta-2,6-dien-1-ol/lactic acid, 3,7-dimethylocta-2,6-dien-1-ol/succinic acid, 3,7-dimethylocta-2,6-dien-1-ol/malic acid, 3,7-dimethylocta-2,6-dien-1-ol/citric acid, 3,7-dimethylocta-2,6-dien-1-ol/acetic acid, 3,7-dimethylocta-2,6-dien-1-ol/formic acid, 3,7-dimethylocta-2,6-dien-1-ol/oxalic acid, 3,7-dimethylocta-2,6-dien-1-ol/uric acid, 3,7-dimethylocta-2,6-dien-1-ol/hydrochloric acid, 3,7-dimethylocta-2,6-dien-1-ol/nitric acid, 3,7-dimethylocta-2,6-dien-1-ol/phosphoric acid, 3,7-dimethylocta-2,6-dien-1-ol/sulphuric acid, 3,7-dimethylocta-2,6-dien-1-ol/boric acid, 3,7-dimethylocta-2,6-dien-1-ol/hydrobromic acid, 3,7-dimethylocta-2,6-dien-1-ol/hydroiodic acid, 3,7-dimethylocta-2,6-dien-1-ol/hydrosulfuric acid, 3,7-dimethylocta-2,6-dien-1-ol/perchloric acid, 3,7-dimethylocta-2,6-dien-1-ol/gallic acid, 3,7-dimethylocta-2,6-dien-1-ol/methyl gallate, 3,7-dimethylocta-2,6-dien-1-ol/3,3,7-dimethylocta-2,6-dien-1-ol/4-dihydroxy benzoic acid, 3,7-dimethylocta-2,6-dien-1-ol/p-hydroxy benzoic acid, 3,7-dimethylocta-2,6-dien-1-ol/vanillic acid, 3,7-dimethylocta-2,6-dien-1-ol/p-coumaric acid, 3,7-dimethylocta-2,6-dien-1-ol/ferulic acid, 3,7-dimethylocta-2,6-dien-1-ol/syringic acid, 3,7-dimethylocta-2,6-dien-1-ol/salicylic acid, 3,7-dimethylocta-2,6-dien-1-ol/luteic acid, 3,7-dimethylocta-2,6-dien-1-ol/eudesmic acid, prop-2-in-1-ol/glycolic acid, prop-2-in-1-ol/lactic acid, prop-2-in-1-ol/succinic acid, prop-2-in-1-ol/malic acid, prop-2-in-1-ol/citric acid, prop-2-in-1-ol/acetic acid, prop-2-in-1-ol/formic acid, prop-2-in-1-ol/oxalic acid, prop-2-in-1-ol/uric acid, prop-2-in-1-ol/hydrochloric acid, prop-2-in-1-ol/nitric acid, prop-2-in-1-ol/phosphoric acid, prop-2-in-1-ol/sulphuric acid, prop-2-in-1-ol/boric acid, prop-2-in-1-ol/hydrobromic acid, prop-2-in-1-ol/hydroiodic acid, prop-2-in-1-ol/hydrosulfuric acid, prop-2-in-1-ol/perchloric acid, prop-2-in-1-ol/gallic acid, prop-2-in-1-ol/methyl gallate, prop-2-in-1-ol/3, prop-2-in-1-ol/4-dihydroxy benzoic acid, prop-2-in-1-ol/p-hydroxy benzoic acid, prop-2-in-1-ol/vanillic acid, prop-2-in-1-ol/p-coumaric acid, prop-2-in-1-ol/ferulic acid, prop-2-in-1-ol/syringic acid, prop-2-in-1-ol/salicylic acid, prop-2-in-1-ol/luteic acid, prop-2-in-1-ol/eudesmic acid, cyclohexane-1,2,3,4,5,6-hexol/glycolic acid, cyclohexane-1,2,3,4,5,6-hexol/lactic acid, cyclohexane-1,2,3,4,5,6-hexol/succinic acid, cyclohexane-1,2,3,4,5,6-hexol/malic acid, cyclohexane-1,2,3,4,5,6-hexol/citric acid, cyclohexane-1,2,3,4,5,6-hexol/acetic acid, cyclohexane-1,2,3,4,5,6-hexol/formic acid, cyclohexane-1,2,3,4,5,6-hexol/oxalic acid, cyclohexane-1,2,3,4,5,6-hexol/uric acid, cyclohexane-1,2,3,4,5,6-hexol/hydrochloric acid, cyclohexane-1,2,3,4,5,6-hexol/nitric acid, cyclohexane-1,2,3,4,5,6-hexol/phosphoric acid, cyclohexane-1,2,3,4,5,6-hexol/sulphuric acid, cyclohexane-1,2,3,4,5,6-hexol/boric acid, cyclohexane-1,2,3,4,5,6-hexol/hydrobromic acid, cyclohexane-1,2,3,4,5,6-hexol/hydroiodic acid, cyclohexane-1,2,3,4,5,6-hexol/hydrosulfuric acid, cyclohexane-1,2,3,4,5,6-hexol/perchloric acid, cyclohexane-1,2,3,4,5,6-hexol/gallic acid, cyclohexane-1,2,3,4,5,6-hexol/methyl gallate, cyclohexane-1,2,3,4,5,6-hexol/3, cyclohexane-1,2,3,4,5,6-hexol/4-dihydroxy benzoic acid, cyclohexane-1,2,3,4,5,6-hexol/p-hydroxy benzoic acid, cyclohexane-1,2,3,4,5,6-hexol/vanillic acid, cyclohexane-1,2,3,4,5,6-hexol/p-coumaric acid, cyclohexane-1,2,3,4,5,6-hexol/ferulic acid, cyclohexane-1,2,3,4,5,6-hexol/syringic acid, cyclohexane-1,2,3,4,5,6-hexol/salicylic acid, cyclohexane-1,2,3,4,5,6-hexol/luteic acid, cyclohexane-1,2,3,4,5,6-hexol/eudesmic acid, 2-(2-propyl)-5-methyl-cyclohexane-1-ol/glycolic acid, 2-(2-propyl)-5-methyl-cyclohexane-1-ol/lactic acid, 2-(2-propyl)-5-methyl-cyclohexane-1-ol/succinic acid, 2-(2-propyl)-5-methyl-cyclohexane-1-ol/malic acid, 2-(2-propyl)-5-methyl-cyclohexane-1-ol/citric acid, 2-(2-propyl)-5-methyl-cyclohexane-1-ol/acetic acid, 2-(2-propyl)-5-methyl-cyclohexane-1-ol/formic acid, 2-(2-propyl)-5-methyl-cyclohexane-1-ol/oxalic acid, 2-(2-propyl)-5-methyl-cyclohexane-1-ol/uric acid, 2-(2-propyl)-5-methyl-cyclohexane-1-ol/hydrochloric acid, 2-(2-propyl)-5-methyl-cyclohexane-1-ol/nitric acid, 2-(2-propyl)-5-methyl-cyclohexane-1-ol/phosphoric acid, 2-(2-propyl)-5-methyl-cyclohexane-1-ol/sulphuric acid, 2-(2-propyl)-5-methyl-cyclohexane-1-ol/boric acid, 2-(2-propyl)-5-methyl-cyclohexane-1-ol/hydrobromic acid, 2-(2-propyl)-5-methyl-cyclohexane-1-ol/hydroiodic acid, 2-(2-propyl)-5-methyl-cyclohexane-1-ol/hydrosulfuric acid, 2-(2-propyl)-5-methyl-cyclohexane-1-ol/perchloric acid, 2-(2-propyl)-5-methyl-cyclohexane-1-ol/gallic acid, 2-(2-propyl)-5-methyl-cyclohexane-1-ol/methyl gallate, 2-(2-propyl)-5-methyl-cyclohexane-1-ol/3,2-(2-propyl)-5-methyl-cyclohexane-1-ol/4-dihydroxy benzoic acid, 2-(2-propyl)-5-methyl-cyclohexane-1-ol/p-hydroxy benzoic acid, 2-(2-propyl)-5-methyl-cyclohexane-1-ol/vanillic acid, 2-(2-propyl)-5-methyl-cyclohexane-1-ol/p-coumaric acid, 2-(2-propyl)-5-methyl-cyclohexane-1-ol/ferulic acid, 2-(2-propyl)-5-methyl-cyclohexane-1-ol/syringic acid, 2-(2-propyl)-5-methyl-cyclohexane-1-ol/salicylic acid, 2-(2-propyl)-5-methyl-cyclohexane-1-ol/luteic acid, and 2-(2-propyl)-5-methyl-cyclohexane-1-ol/eudesmic acid.

In still another embodiment, a composition is provided that that inactivates or inhibits replication of a virus, where the composition comprises one or more alcohols selected from the group consisting of butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol; an acid; and a pharmaceutically acceptable carrier; where the pH of said composition is about pH 4.5 or lower. In various embodiments this composition further comprises one or more agents selected from the group consisting of an antioxidant phenolic compound, a tannin, a dicarboxylic acids or derivative thereof, mandelic acid, butylated hydroxy toluene (BHT), an extract from the plant Prunella vulgaris, and vitamin C. In certain embodiments this composition comprises an antioxidant phenolic compound selected from the group consisting of phenol, resorcinol, and resveratrol. In certain embodiments this composition comprises a dicarboxylic acid selected from the group consisting of succinic acid, and tartaric acid. In certain embodiments the acid in this composition comprises one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid. In certain embodiments the alcohol/acid combination comprises an alcohol/acid pair selected from the group consisting of butyl alcohol/glycolic acid, butyl alcohol/lactic acid, butyl alcohol/succinic acid, butyl alcohol/malic acid, butyl alcohol/citric acid, butyl alcohol/acetic acid, butyl alcohol/formic acid, butyl alcohol/oxalic acid, butyl alcohol/uric acid, butyl alcohol/hydrochloric acid, butyl alcohol/nitric acid, butyl alcohol/phosphoric acid, butyl alcohol/sulphuric acid, butyl alcohol/boric acid, butyl alcohol/hydrobromic acid, butyl alcohol/hydroiodic acid, butyl alcohol/hydrosulfuric acid, butyl alcohol/perchloric acid, butyl alcohol/gallic acid, butyl alcohol/methyl gallate, butyl alcohol/3,4-dihydroxy benzoic acid, butyl alcohol/p-hydroxy benzoic acid, butyl alcohol/vanillic acid, butyl alcohol/p-coumaric acid, butyl alcohol/ferulic acid, butyl alcohol/syringic acid, butyl alcohol/salicylic acid, butyl alcohol/luteic acid, butyl alcohol/eudesmic acid, n-butanol/glycolic acid, n-butanol/lactic acid, n-butanol/succinic acid, n-butanol/malic acid, n-butanol/citric acid, n-butanol/acetic acid, n-butanol/formic acid, n-butanol/oxalic acid, n-butanol/uric acid, n-butanol/hydrochloric acid, n-butanol/nitric acid, n-butanol/phosphoric acid, n-butanol/sulphuric acid, n-butanol/boric acid, n-butanol/hydrobromic acid, n-butanol/hydroiodic acid, n-butanol/hydrosulfuric acid, n-butanol/perchloric acid, n-butanol/gallic acid, n-butanol/methyl gallate, n-butanol/3,4-dihydroxy benzoic acid, n-butanol/p-hydroxy benzoic acid, n-butanol/vanillic acid, n-butanol/p-coumaric acid, n-butanol/ferulic acid, n-butanol/syringic acid, n-butanol/salicylic acid, n-butanol/luteic acid, n-butanol/eudesmic acid, sec-butanol/glycolic acid, sec-butanol/lactic acid, sec-butanol/succinic acid, sec-butanol/malic acid, sec-butanol/citric acid, sec-butanol/acetic acid, sec-butanol/formic acid, sec-butanol/oxalic acid, sec-butanol/uric acid, sec-butanol/hydrochloric acid, sec-butanol/nitric acid, sec-butanol/phosphoric acid, sec-butanol/sulphuric acid, sec-butanol/boric acid, sec-butanol/hydrobromic acid, sec-butanol/hydroiodic acid, sec-butanol/hydrosulfuric acid, sec-butanol/perchloric acid, sec-butanol/gallic acid, sec-butanol/methyl gallate, sec-butanol/3,4-dihydroxy benzoic acid, sec-butanol/p-hydroxy benzoic acid, sec-butanol/vanillic acid, sec-butanol/p-coumaric acid, sec-butanol/ferulic acid, sec-butanol/syringic acid, sec-butanol/salicylic acid, sec-butanol/luteic acid, sec-butanol/eudesmic acid, isobutanol/glycolic acid, isobutanol/lactic acid, isobutanol/succinic acid, isobutanol/malic acid, isobutanol/citric acid, isobutanol/acetic acid, isobutanol/formic acid, isobutanol/oxalic acid, isobutanol/uric acid, isobutanol/hydrochloric acid, isobutanol/nitric acid, isobutanol/phosphoric acid, isobutanol/sulphuric acid, isobutanol/boric acid, isobutanol/hydrobromic acid, isobutanol/hydroiodic acid, isobutanol/hydrosulfuric acid, isobutanol/perchloric acid, isobutanol/gallic acid, isobutanol/methyl gallate, isobutanol/3,4-dihydroxy benzoic acid, isobutanol/p-hydroxy benzoic acid, isobutanol/vanillic acid, isobutanol/p-coumaric acid, isobutanol/ferulic acid, isobutanol/syringic acid, isobutanol/salicylic acid, isobutanol/luteic acid, isobutanol/eudesmic acid, tert-butanol/glycolic acid, tert-butanol/lactic acid, tert-butanol/succinic acid, tert-butanol/malic acid, tert-butanol/citric acid, tert-butanol/acetic acid, tert-butanol/formic acid, tert-butanol/oxalic acid, tert-butanol/uric acid, tert-butanol/hydrochloric acid, tert-butanol/nitric acid, tert-butanol/phosphoric acid, tert-butanol/sulphuric acid, tert-butanol/boric acid, tert-butanol/hydrobromic acid, tert-butanol/hydroiodic acid, tert-butanol/hydrosulfuric acid, tert-butanol/perchloric acid, tert-butanol/gallic acid, tert-butanol/methyl gallate, tert-butanol/3,4-dihydroxy benzoic acid, tert-butanol/p-hydroxy benzoic acid, tert-butanol/vanillic acid, tert-butanol/p-coumaric acid, tert-butanol/ferulic acid, tert-butanol/syringic acid, tert-butanol/salicylic acid, tert-butanol/luteic acid, tert-butanol/eudesmic acid, pentanol/glycolic acid, pentanol/lactic acid, pentanol/succinic acid, pentanol/malic acid, pentanol/citric acid, pentanol/acetic acid, pentanol/formic acid, pentanol/oxalic acid, pentanol/uric acid, pentanol/hydrochloric acid, pentanol/nitric acid, pentanol/phosphoric acid, pentanol/sulphuric acid, pentanol/boric acid, pentanol/hydrobromic acid, pentanol/hydroiodic acid, pentanol/hydrosulfuric acid, pentanol/perchloric acid, pentanol/gallic acid, pentanol/methyl gallate, pentanol/3,4-dihydroxy benzoic acid, pentanol/p-hydroxy benzoic acid, pentanol/vanillic acid, pentanol/p-coumaric acid, pentanol/ferulic acid, pentanol/syringic acid, pentanol/salicylic acid, pentanol/luteic acid, pentanol/eudesmic acid, hexadecan-1-ol/glycolic acid, hexadecan-1-ol/lactic acid, hexadecan-1-ol/succinic acid, hexadecan-1-ol/malic acid, hexadecan-1-ol/citric acid, hexadecan-1-ol/acetic acid, hexadecan-1-ol/formic acid, hexadecan-1-ol/oxalic acid, hexadecan-1-ol/uric acid, hexadecan-1-ol/hydrochloric acid, hexadecan-1-ol/nitric acid, hexadecan-1-ol/phosphoric acid, hexadecan-1-ol/sulphuric acid, hexadecan-1-ol/boric acid, hexadecan-1-ol/hydrobromic acid, hexadecan-1-ol/hydroiodic acid, hexadecan-1-ol/hydrosulfuric acid, hexadecan-1-ol/perchloric acid, hexadecan-1-ol/gallic acid, hexadecan-1-ol/methyl gallate, hexadecan-1-ol/3,4-dihydroxy benzoic acid, hexadecan-1-ol/p-hydroxy benzoic acid, hexadecan-1-ol/vanillic acid, hexadecan-1-ol/p-coumaric acid, hexadecan-1-ol/ferulic acid, hexadecan-1-ol/syringic acid, hexadecan-1-ol/salicylic acid, hexadecan-1-ol/luteic acid, hexadecan-1-ol/eudesmic acid, ethane-1,2-diol/glycolic acid, ethane-1,2-diol/lactic acid, ethane-1,2-diol/succinic acid, ethane-1,2-diol/malic acid, ethane-1,2-diol/citric acid, ethane-1,2-diol/acetic acid, ethane-1,2-diol/formic acid, ethane-1,2-diol/oxalic acid, ethane-1,2-diol/uric acid, ethane-1,2-diol/hydrochloric acid, ethane-1,2-diol/nitric acid, ethane-1,2-diol/phosphoric acid, ethane-1,2-diol/sulphuric acid, ethane-1,2-diol/boric acid, ethane-1,2-diol/hydrobromic acid, ethane-1,2-diol/hydroiodic acid, ethane-1,2-diol/hydrosulfuric acid, ethane-1,2-diol/perchloric acid, ethane-1,2-diol/gallic acid, ethane-1,2-diol/methyl gallate, ethane-1,2-diol/3,4-dihydroxy benzoic acid, ethane-1,2-diol/p-hydroxy benzoic acid, ethane-1,2-diol/vanillic acid, ethane-1,2-diol/p-coumaric acid, ethane-1,2-diol/ferulic acid, ethane-1,2-diol/syringic acid, ethane-1,2-diol/salicylic acid, ethane-1,2-diol/luteic acid, ethane-1,2-diol/eudesmic acid, propane-1,2-diol/glycolic acid, propane-1,2-diol/lactic acid, propane-1,2-diol/succinic acid, propane-1,2-diol/malic acid, propane-1,2-diol/citric acid, propane-1,2-diol/acetic acid, propane-1,2-diol/formic acid, propane-1,2-diol/oxalic acid, propane-1,2-diol/uric acid, propane-1,2-diol/hydrochloric acid, propane-1,2-diol/nitric acid, propane-1,2-diol/phosphoric acid, propane-1,2-diol/sulphuric acid, propane-1,2-diol/boric acid, propane-1,2-diol/hydrobromic acid, propane-1,2-diol/hydroiodic acid, propane-1,2-diol/hydrosulfuric acid, propane-1,2-diol/perchloric acid, propane-1,2-diol/gallic acid, propane-1,2-diol/methyl gallate, propane-1,2-diol/3,4-dihydroxy benzoic acid, propane-1,2-diol/p-hydroxy benzoic acid, propane-1,2-diol/vanillic acid, propane-1,2-diol/p-coumaric acid, propane-1,2-diol/ferulic acid, propane-1,2-diol/syringic acid, propane-1,2-diol/salicylic acid, propane-1,2-diol/luteic acid, propane-1,2-diol/eudesmic acid, propane-1,2,3-triol/glycolic acid, propane-1,2,3-triol/lactic acid, propane-1,2,3-triol/succinic acid, propane-1,2,3-triol/malic acid, propane-1,2,3-triol/citric acid, propane-1,2,3-triol/acetic acid, propane-1,2,3-triol/formic acid, propane-1,2,3-triol/oxalic acid, propane-1,2,3-triol/uric acid, propane-1,2,3-triol/hydrochloric acid, propane-1,2,3-triol/nitric acid, propane-1,2,3-triol/phosphoric acid, propane-1,2,3-triol/sulphuric acid, propane-1,2,3-triol/boric acid, propane-1,2,3-triol/hydrobromic acid, propane-1,2,3-triol/hydroiodic acid, propane-1,2,3-triol/hydrosulfuric acid, propane-1,2,3-triol/perchloric acid, propane-1,2,3-triol/gallic acid, propane-1,2,3-triol/methyl gallate, propane-1,2,3-triol/3,4-dihydroxy benzoic acid, propane-1,2,3-triol/p-hydroxy benzoic acid, propane-1,2,3-triol/vanillic acid, propane-1,2,3-triol/p-coumaric acid, propane-1,2,3-triol/ferulic acid, propane-1,2,3-triol/syringic acid, propane-1,2,3-triol/salicylic acid, propane-1,2,3-triol/luteic acid, propane-1,2,3-triol/eudesmic acid, butane-1,2,3,4-tetraol/glycolic acid, butane-1,2,3,4-tetraol/lactic acid, butane-1,2,3,4-tetraol/succinic acid, butane-1,2,3,4-tetraol/malic acid, butane-1,2,3,4-tetraol/citric acid, butane-1,2,3,4-tetraol/acetic acid, butane-1,2,3,4-tetraol/formic acid, butane-1,2,3,4-tetraol/oxalic acid, butane-1,2,3,4-tetraol/uric acid, butane-1,2,3,4-tetraol/hydrochloric acid, butane-1,2,3,4-tetraol/nitric acid, butane-1,2,3,4-tetraol/phosphoric acid, butane-1,2,3,4-tetraol/sulphuric acid, butane-1,2,3,4-tetraol/boric acid, butane-1,2,3,4-tetraol/hydrobromic acid, butane-1,2,3,4-tetraol/hydroiodic acid, butane-1,2,3,4-tetraol/hydrosulfuric acid, butane-1,2,3,4-tetraol/perchloric acid, butane-1,2,3,4-tetraol/gallic acid, butane-1,2,3,4-tetraol/methyl gallate, butane-1,2,3,4-tetraol/3,4-dihydroxy benzoic acid, butane-1,2,3,4-tetraol/p-hydroxy benzoic acid, butane-1,2,3,4-tetraol/vanillic acid, butane-1,2,3,4-tetraol/p-coumaric acid, butane-1,2,3,4-tetraol/ferulic acid, butane-1,2,3,4-tetraol/syringic acid, butane-1,2,3,4-tetraol/salicylic acid, butane-1,2,3,4-tetraol/luteic acid, butane-1,2,3,4-tetraol/eudesmic acid, pentane-1,2,3,4,5-pentol/glycolic acid, pentane-1,2,3,4,5-pentol/lactic acid, pentane-1,2,3,4,5-pentol/succinic acid, pentane-1,2,3,4,5-pentol/malic acid, pentane-1,2,3,4,5-pentol/citric acid, pentane-1,2,3,4,5-pentol/acetic acid, pentane-1,2,3,4,5-pentol/formic acid, pentane-1,2,3,4,5-pentol/oxalic acid, pentane-1,2,3,4,5-pentol/uric acid, pentane-1,2,3,4,5-pentol/hydrochloric acid, pentane-1,2,3,4,5-pentol/nitric acid, pentane-1,2,3,4,5-pentol/phosphoric acid, pentane-1,2,3,4,5-pentol/sulphuric acid, pentane-1,2,3,4,5-pentol/boric acid, pentane-1,2,3,4,5-pentol/hydrobromic acid, pentane-1,2,3,4,5-pentol/hydroiodic acid, pentane-1,2,3,4,5-pentol/hydrosulfuric acid, pentane-1,2,3,4,5-pentol/perchloric acid, pentane-1,2,3,4,5-pentol/gallic acid, pentane-1,2,3,4,5-pentol/methyl gallate, pentane-1,2,3,4,5-pentol/3,4-dihydroxy benzoic acid, pentane-1,2,3,4,5-pentol/p-hydroxy benzoic acid, pentane-1,2,3,4,5-pentol/vanillic acid, pentane-1,2,3,4,5-pentol/p-coumaric acid, pentane-1,2,3,4,5-pentol/ferulic acid, pentane-1,2,3,4,5-pentol/syringic acid, pentane-1,2,3,4,5-pentol/salicylic acid, pentane-1,2,3,4,5-pentol/luteic acid, pentane-1,2,3,4,5-pentol/eudesmic acid, hexane-1,2,3,4,5,6-hexol/glycolic acid, hexane-1,2,3,4,5,6-hexol/lactic acid, hexane-1,2,3,4,5,6-hexol/succinic acid, hexane-1,2,3,4,5,6-hexol/malic acid, hexane-1,2,3,4,5,6-hexol/citric acid, hexane-1,2,3,4,5,6-hexol/acetic acid, hexane-1,2,3,4,5,6-hexol/formic acid, hexane-1,2,3,4,5,6-hexol/oxalic acid, hexane-1,2,3,4,5,6-hexol/uric acid, hexane-1,2,3,4,5,6-hexol/hydrochloric acid, hexane-1,2,3,4,5,6-hexol/nitric acid, hexane-1,2,3,4,5,6-hexol/phosphoric acid, hexane-1,2,3,4,5,6-hexol/sulphuric acid, hexane-1,2,3,4,5,6-hexol/boric acid, hexane-1,2,3,4,5,6-hexol/hydrobromic acid, hexane-1,2,3,4,5,6-hexol/hydroiodic acid, hexane-1,2,3,4,5,6-hexol/hydrosulfuric acid, hexane-1,2,3,4,5,6-hexol/perchloric acid, hexane-1,2,3,4,5,6-hexol/gallic acid, hexane-1,2,3,4,5,6-hexol/methyl gallate, hexane-1,2,3,4,5,6-hexol/3,4-dihydroxy benzoic acid, hexane-1,2,3,4,5,6-hexol/p-hydroxy benzoic acid, hexane-1,2,3,4,5,6-hexol/vanillic acid, hexane-1,2,3,4,5,6-hexol/p-coumaric acid, hexane-1,2,3,4,5,6-hexol/ferulic acid, hexane-1,2,3,4,5,6-hexol/syringic acid, hexane-1,2,3,4,5,6-hexol/salicylic acid, hexane-1,2,3,4,5,6-hexol/luteic acid, hexane-1,2,3,4,5,6-hexol/eudesmic acid, heptane-1,2,3,4,5,6,7-heptol/glycolic acid, heptane-1,2,3,4,5,6,7-heptol/lactic acid, heptane-1,2,3,4,5,6,7-heptol/succinic acid, heptane-1,2,3,4,5,6,7-heptol/malic acid, heptane-1,2,3,4,5,6,7-heptol/citric acid, heptane-1,2,3,4,5,6,7-heptol/acetic acid, heptane-1,2,3,4,5,6,7-heptol/formic acid, heptane-1,2,3,4,5,6,7-heptol/oxalic acid, heptane-1,2,3,4,5,6,7-heptol/uric acid, heptane-1,2,3,4,5,6,7-heptol/hydrochloric acid, heptane-1,2,3,4,5,6,7-heptol/nitric acid, heptane-1,2,3,4,5,6,7-heptol/phosphoric acid, heptane-1,2,3,4,5,6,7-heptol/sulphuric acid, heptane-1,2,3,4,5,6,7-heptol/boric acid, heptane-1,2,3,4,5,6,7-heptol/hydrobromic acid, heptane-1,2,3,4,5,6,7-heptol/hydroiodic acid, heptane-1,2,3,4,5,6,7-heptol/hydrosulfuric acid, heptane-1,2,3,4,5,6,7-heptol/perchloric acid, heptane-1,2,3,4,5,6,7-heptol/gallic acid, heptane-1,2,3,4,5,6,7-heptol/methyl gallate, heptane-1,2,3,4,5,6,7-heptol/3,4-dihydroxy benzoic acid, heptane-1,2,3,4,5,6,7-heptol/p-hydroxy benzoic acid, heptane-1,2,3,4,5,6,7-heptol/vanillic acid, heptane-1,2,3,4,5,6,7-heptol/p-coumaric acid, heptane-1,2,3,4,5,6,7-heptol/ferulic acid, heptane-1,2,3,4,5,6,7-heptol/syringic acid, heptane-1,2,3,4,5,6,7-heptol/salicylic acid, heptane-1,2,3,4,5,6,7-heptol/luteic acid, heptane-1,2,3,4,5,6,7-heptol/eudesmic acid, prop-2-ene-1-ol/glycolic acid, prop-2-ene-1-ol/lactic acid, prop-2-ene-1-ol/succinic acid, prop-2-ene-1-ol/malic acid, prop-2-ene-1-ol/citric acid, prop-2-ene-1-ol/acetic acid, prop-2-ene-1-ol/formic acid, prop-2-ene-1-ol/oxalic acid, prop-2-ene-1-ol/uric acid, prop-2-ene-1-ol/hydrochloric acid, prop-2-ene-1-ol/nitric acid, prop-2-ene-1-ol/phosphoric acid, prop-2-ene-1-ol/sulphuric acid, prop-2-ene-1-ol/boric acid, prop-2-ene-1-ol/hydrobromic acid, prop-2-ene-1-ol/hydroiodic acid, prop-2-ene-1-ol/hydrosulfuric acid, prop-2-ene-1-ol/perchloric acid, prop-2-ene-1-ol/gallic acid, prop-2-ene-1-ol/methyl gallate, prop-2-ene-1-ol/3,4-dihydroxy benzoic acid, prop-2-ene-1-ol/p-hydroxy benzoic acid, prop-2-ene-1-ol/vanillic acid, prop-2-ene-1-ol/p-coumaric acid, prop-2-ene-1-ol/ferulic acid, prop-2-ene-1-ol/syringic acid, prop-2-ene-1-ol/salicylic acid, prop-2-ene-1-ol/luteic acid, prop-2-ene-1-ol/eudesmic acid, 3,7-dimethylocta-2,6-dien-1-ol/glycolic acid, 3,7-dimethylocta-2,6-dien-1-ol/lactic acid, 3,7-dimethylocta-2,6-dien-1-ol/succinic acid, 3,7-dimethylocta-2,6-dien-1-ol/malic acid, 3,7-dimethylocta-2,6-dien-1-ol/citric acid, 3,7-dimethylocta-2,6-dien-1-ol/acetic acid, 3,7-dimethylocta-2,6-dien-1-ol/formic acid, 3,7-dimethylocta-2,6-dien-1-ol/oxalic acid, 3,7-dimethylocta-2,6-dien-1-ol/uric acid, 3,7-dimethylocta-2,6-dien-1-ol/hydrochloric acid, 3,7-dimethylocta-2,6-dien-1-ol/nitric acid, 3,7-dimethylocta-2,6-dien-1-ol/phosphoric acid, 3,7-dimethylocta-2,6-dien-1-ol/sulphuric acid, 3,7-dimethylocta-2,6-dien-1-ol/boric acid, 3,7-dimethylocta-2,6-dien-1-ol/hydrobromic acid, 3,7-dimethylocta-2,6-dien-1-ol/hydroiodic acid, 3,7-dimethylocta-2,6-dien-1-ol/hydrosulfuric acid, 3,7-dimethylocta-2,6-dien-1-ol/perchloric acid, 3,7-dimethylocta-2,6-dien-1-ol/gallic acid, 3,7-dimethylocta-2,6-dien-1-ol/methyl gallate, 3,7-dimethylocta-2,6-dien-1-ol/3,4-dihydroxy benzoic acid, 3,7-dimethylocta-2,6-dien-1-ol/p-hydroxy benzoic acid, 3,7-dimethylocta-2,6-dien-1-ol/vanillic acid, 3,7-dimethylocta-2,6-dien-1-ol/p-coumaric acid, 3,7-dimethylocta-2,6-dien-1-ol/ferulic acid, 3,7-dimethylocta-2,6-dien-1-ol/syringic acid, 3,7-dimethylocta-2,6-dien-1-ol/salicylic acid, 3,7-dimethylocta-2,6-dien-1-ol/luteic acid, 3,7-dimethylocta-2,6-dien-1-ol/eudesmic acid, prop-2-in-1-ol/glycolic acid, prop-2-in-1-ol/lactic acid, prop-2-in-1-ol/succinic acid, prop-2-in-1-ol/malic acid, prop-2-in-1-ol/citric acid, prop-2-in-1-ol/acetic acid, prop-2-in-1-ol/formic acid, prop-2-in-1-ol/oxalic acid, prop-2-in-1-ol/uric acid, prop-2-in-1-ol/hydrochloric acid, prop-2-in-1-ol/nitric acid, prop-2-in-1-ol/phosphoric acid, prop-2-in-1-ol/sulphuric acid, prop-2-in-1-ol/boric acid, prop-2-in-1-ol/hydrobromic acid, prop-2-in-1-ol/hydroiodic acid, prop-2-in-1-ol/hydrosulfuric acid, prop-2-in-1-ol/perchloric acid, prop-2-in-1-ol/gallic acid, prop-2-in-1-ol/methyl gallate, prop-2-in-1-ol/3,4-dihydroxy benzoic acid, prop-2-in-1-ol/p-hydroxy benzoic acid, prop-2-in-1-ol/vanillic acid, prop-2-in-1-ol/p-coumaric acid, prop-2-in-1-ol/ferulic acid, prop-2-in-1-ol/syringic acid, prop-2-in-1-ol/salicylic acid, prop-2-in-1-ol/luteic acid, prop-2-in-1-ol/eudesmic acid, cyclohexane-1,2,3,4,5,6-hexol/glycolic acid, cyclohexane-1,2,3,4,5,6-hexol/lactic acid, cyclohexane-1,2,3,4,5,6-hexol/succinic acid, cyclohexane-1,2,3,4,5,6-hexol/malic acid, cyclohexane-1,2,3,4,5,6-hexol/citric acid, cyclohexane-1,2,3,4,5,6-hexol/acetic acid, cyclohexane-1,2,3,4,5,6-hexol/formic acid, cyclohexane-1,2,3,4,5,6-hexol/oxalic acid, cyclohexane-1,2,3,4,5,6-hexol/uric acid, cyclohexane-1,2,3,4,5,6-hexol/hydrochloric acid, cyclohexane-1,2,3,4,5,6-hexol/nitric acid, cyclohexane-1,2,3,4,5,6-hexol/phosphoric acid, cyclohexane-1,2,3,4,5,6-hexol/sulphuric acid, cyclohexane-1,2,3,4,5,6-hexol/boric acid, cyclohexane-1,2,3,4,5,6-hexol/hydrobromic acid, cyclohexane-1,2,3,4,5,6-hexol/hydroiodic acid, cyclohexane-1,2,3,4,5,6-hexol/hydrosulfuric acid, cyclohexane-1,2,3,4,5,6-hexol/perchloric acid, cyclohexane-1,2,3,4,5,6-hexol/gallic acid, cyclohexane-1,2,3,4,5,6-hexol/methyl gallate, cyclohexane-1,2,3,4,5,6-hexol/3,4-dihydroxy benzoic acid, cyclohexane-1,2,3,4,5,6-hexol/p-hydroxy benzoic acid, cyclohexane-1,2,3,4,5,6-hexol/vanillic acid, cyclohexane-1,2,3,4,5,6-hexol/p-coumaric acid, cyclohexane-1,2,3,4,5,6-hexol/ferulic acid, cyclohexane-1,2,3,4,5,6-hexol/syringic acid, cyclohexane-1,2,3,4,5,6-hexol/salicylic acid, cyclohexane-1,2,3,4,5,6-hexol/luteic acid, cyclohexane-1,2,3,4,5,6-hexol/eudesmic acid, 2-(2-propyl)-5-methyl-cyclohexane-1-ol/glycolic acid, 2-(2-propyl)-5-methyl-cyclohexane-1-ol/lactic acid, 2-(2-propyl)-5-methyl-cyclohexane-1-ol/succinic acid, 2-(2-propyl)-5-methyl-cyclohexane-1-ol/malic acid, 2-(2-propyl)-5-methyl-cyclohexane-1-ol/citric acid, 2-(2-propyl)-5-methyl-cyclohexane-1-ol/acetic acid, 2-(2-propyl)-5-methyl-cyclohexane-1-ol/formic acid, 2-(2-propyl)-5-methyl-cyclohexane-1-ol/oxalic acid, 2-(2-propyl)-5-methyl-cyclohexane-1-ol/uric acid, 2-(2-propyl)-5-methyl-cyclohexane-1-ol/hydrochloric acid, 2-(2-propyl)-5-methyl-cyclohexane-1-ol/nitric acid, 2-(2-propyl)-5-methyl-cyclohexane-1-ol/phosphoric acid, 2-(2-propyl)-5-methyl-cyclohexane-1-ol/sulphuric acid, 2-(2-propyl)-5-methyl-cyclohexane-1-ol/boric acid, 2-(2-propyl)-5-methyl-cyclohexane-1-ol/hydrobromic acid, 2-(2-propyl)-5-methyl-cyclohexane-1-ol/hydroiodic acid, 2-(2-propyl)-5-methyl-cyclohexane-1-ol/hydrosulfuric acid, 2-(2-propyl)-5-methyl-cyclohexane-1-ol/perchloric acid, 2-(2-propyl)-5-methyl-cyclohexane-1-ol/gallic acid, 2-(2-propyl)-5-methyl-cyclohexane-1-ol/methyl gallate, 2-(2-propyl)-5-methyl-cyclohexane-1-ol/3,4-dihydroxy benzoic acid, 2-(2-propyl)-5-methyl-cyclohexane-1-ol/p-hydroxy benzoic acid, 2-(2-propyl)-5-methyl-cyclohexane-1-ol/vanillic acid, 2-(2-propyl)-5-methyl-cyclohexane-1-ol/p-coumaric acid, 2-(2-propyl)-5-methyl-cyclohexane-1-ol/ferulic acid, 2-(2-propyl)-5-methyl-cyclohexane-1-ol/syringic acid, 2-(2-propyl)-5-methyl-cyclohexane-1-ol/salicylic acid, 2-(2-propyl)-5-methyl-cyclohexane-1-ol/luteic acid, and 2-(2-propyl)-5-methyl-cyclohexane-1-ol/eudesmic acid.

In various embodiments of any of the foregoing compositions, the alcohol is ranges from about 0.2% by volume up to about 40% by volume of the composition. In certain embodiments the alcohol ranges from about 0.5% by volume up to about 30% or up to about 20% by volume of the composition. In certain embodiments the alcohol ranges from about 1% by volume up to about 15% by volume or from about 2% by volume up to about 13% by volume or from about 2% up to about 10% by volume of the composition. In one embodiment of any of the foregoing compositions the alcohol comprises about 5% by volume of the composition. In one embodiment of any of the foregoing compositions the alcohol comprises about 6% by volume of the composition. In one embodiment of any of the foregoing compositions the alcohol comprises about 7% by volume of the composition. In one embodiment of any of the foregoing compositions the alcohol comprises about 8% by volume of the composition. In one embodiment of any of the foregoing compositions the alcohol comprises about 9% by volume of the composition. In one embodiment of any of the foregoing compositions the alcohol comprises about 10% by volume of the composition. In one embodiment of any of the foregoing compositions the alcohol comprises about 11% by volume of the composition. In one embodiment of any of the foregoing compositions the alcohol comprises about 12% by volume of the composition. In one embodiment of any of the foregoing compositions the alcohol comprises about 13% by volume of the composition. In one embodiment of any of the foregoing compositions the alcohol comprises about 14% by volume of the composition. In one embodiment of any of the foregoing compositions the alcohol comprises about 15% by volume of the composition.

In one embodiment of any of the foregoing compositions the alcohol comprises about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 38%, or about 38%, or about 39%, or about 40% by volume of the composition.

In certain embodiments the acid, is present at about 0.1% to about 50%, or about 0.1% to about 40%, or about 0.1% to about 30%, or about 0.1% to about 20%, or about 0.1% to about 15%, or about 0.1% to about 10%, or about 0.1% to about 5%, or about 0.2% to about 4%, or about 0.3% to about 3%, or about 0.4% to about 2% by weight. In certain embodiments the acid is present at about 0.1, or about 0.2%, or about 0.3%, or about 0.4%, or about 0.5%, or about 0.6%, or about 0.7%, or about 0.8%, or about 0.9%, or about 1.0%, or about 1.1%, or about 1.2%, or about 1.3%, or about 1.4%, or about 1.5%, or about 1.6%, or about 1.7%, or about 1.8%, or about 1.9%, or about 2.0% by weight of the composition.

In certain embodiments, the acid is present at about 0.1%, or about or 0.2%, or about 0.3%, or about 0.4%, or about 0.5%, or about 0.6%, or about 0.7%, or about 0.8%, or about 0.9%, or about 1.0%, or about 1.1%, or about 1.2%, or about 1.3%, or about 1.4%, or about 1.5%, or about 1.6%, or about 1.7%, or about 1.8.%, or about 1.9%, or about 2.0%, or about 2.1%, or about 2.2%, or about 2.3%, or about 2.4%, or about 2.5%, or about 3%, or about 3.5%, or about 4%, or about 4.5%, or about 5%, or about 5.5%, or about 6%, or about 6.5%, or about 7%, or about 7.5%, or about 8%, or about 8.5%, or about 89%, or about 9.5%, or about 10%, or about 11%, or about 12%, or about 13%, or about 14%, or about 15%, or about 16%, or about 17%, or about 18%, or about 19%, or about 20%, or about 21%, or about 22%, or about 23,24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, by weight of the composition.

In certain embodiments the acid is present in an amount sufficient to adjust the pH of the composition to about pH 4.5 or lower, or to about pH 4.0, or to about pH 3.5 or lower, or to about pH 3.0 or lower, or to about pH 2.5 or lower. In certain embodiments the acid is present in an amount sufficient to adjust the pH of the composition to about pH 4.5 or less, or about pH 4 or less, or about pH 3.5 or less, or about pH 3 or less, or about pH 2.5 or less, or about pH 2. In certain embodiments the acid is present in an amount sufficient to adjust the pH of the composition to about pH 1.5 or pH 2 up to about pH 4.5, or up to about pH 4, or up to about pH 3.5, or up to about pH 3, or up to about pH 2.5.

As described below, in various embodiments, the composition is formulated as a fluid, e.g., an aqueous solution or suspension including for example a tincture, a roll-on, a spray, a gel, an ointment, a cream, a salve, a lotion, a lip balm, a foam, a spray, and aerosol.

The compositions described herein typically contain ingredients, e.g. an alcohol and an acid, that individually are not potent antivirals when applied topically to a site of infection. For example, it is known that certain organic acids such as dicarboxylic acids are virucidal in vitro but are ineffective when applied topically. Surprisingly, however, a synergistic antiviral effect is noted when these ingredients (i.e., an acid and an alcohol as described herein) are used in combination in the compositions described herein. For example, it was surprisingly discovered that when an effective amount of a virucidal composition described herein, having a pH at or below about pH 4.5 was topically applied to a lesion or to the site of a potential lesion caused by a virus such as HSV-1, the virus was substantially inactivated, thereby interrupting and preventing the spread of the virus. Thus, it was surprisingly and unexpectedly discovered that the compositions described herein provide effective topical virucidal compositions.

In one illustrative example, the topical compositions described herein are found to be topically active in vivo against HSV-1 (causing cold sores and fever blisters), HSV-2 (causing genital herpes), and herpes zoster (causing shingles). It is believed that these compositions will also be active against other viruses in the Herpesviridae family, that are similar in structure and/or mechanism of infection, including, for example, the varicella virus (causing chicken pox).

It is well known in the art that ethanol or propanols alone have no virucidal activity against enveloped viruses. While not wishing to be bound by any particular theory, it is believed that the alcohol(s) used in the compositions described herein facilitate penetration of the acid through the outer dermal layer to the site of viral replication. In any case, a synergy is observed between the acid and the alcohol, in that it is believed that both must be used to achieve significant topical virucidal activity.

A further advantage of the compositions of described herein is that the components (e.g., relatively short chain alcohols and various acids) avoid the insolubility problems as well as the unpleasant odors associated with other antiviral agents. alcohols having longer chain lengths

While not wishing to be bound by any theory, it is believed that the compositions described herein remain active for over 24 hours in the dermal/epidermal junctions, and display effective antiviral activity over such a time period.

### Topical Formulations.

In various embodiments the compositions described herein are formulated for topical administration to a mammal (e.g., to a human or to a non-human mammal). Methods that are customary in industrial pharmaceutics can be employed to produce topical formulations. For this, the active compounds (e.g., the alcohol and acid) are processed, preferably together with a pharmaceutically acceptable carrier particularly a carrier suitable for application on the skin and/or mucosa.

It will be recognized that the various active components (e.g., alcohol(s), acids(s), additional components are water soluble and, in certain embodiments, can simply be admixed in an aqueous formulation and/or as a tincture.

In certain embodiments the composition is formulated as a solution for topical application, that is, a liquid formulation comprising the alcohol the acid, and in certain embodiments, the additional component.

Other suitable forms include, but are not limited to, semi-solid or solid forms comprising a carrier indigenous to topical application and typically having a dynamic viscosity preferably greater than that of water, provided that the carrier does not deleteriously react with the acid, the alcohol, or when present the additional component(s) in the composition. Suitable formulations include, but are not limited to, lip balms, suspensions, emulsions, creams, ointments, powders, liniments, salves and the like. If desired, these may be sterilized or mixed with auxiliary agents, e.g., preservatives, stabilizers, wetting agents, buffers or salts for influencing osmotic pressure and the like.

In certain embodiments vehicles for semi-solid or solid forms topical preparations include, but are not limited to ointment bases, e.g., polyethylene glycol-1000 (pEG-1000); creams, e.g., HEB cream; and gels, e.g., K-Y gel; as well as petroleum jelly and the like. These topical preparations may also contain emollients, perfumes, and/or pigments to enhance their acceptability for various usages, provided that the additives do not deleteriously react with the acid or the alcohol in the composition.

Also suitable for topical application are sprayable aerosol preparations wherein the composition, preferably in combination with a solid or liquid inert carrier material, is packaged in a squeeze bottle or in admixture with a pressurized volatile, normally gaseous propellant, e.g., a Freon (chlorofluorocarbon), or environmentally acceptable volatile propellant. Such compositions can be used for application to environmental surfaces, e.g., examining tables, toilet seats and the like, and/or for application to the skin or to mucous membranes. The aerosol or spray preparations can contain solvents, buffers, surfactants, perfumes, and/or antioxidants in addition to the virucidal compounds of the invention.

In various embodiments the compositions described herein can be employed in mixture with conventional excipients, i.e., pharmaceutically acceptable organic or inorganic carrier substances suitable for topical application which do not deleteriously react with the acid or the alcohol in the composition. Suitable pharmaceutically acceptable carriers include but are not limited to water, salt solutions, buffer solutions (e.g., phosphate buffers, citrate buffers, etc.), alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxymethylcellulose, polyvinyl pyrrolidone, lubricants, preservatives, stabilizers, wetting agents, emulsifiers, salts for influencing osmotic pressure, buffers, coloring, flavoring and/or aromatic substances and the like which do not deleteriously react with the acid or the alcohol in the composition.

Customary emulsions, gels, ointments, creams of the mixed-phase or amphiphilic emulsion systems (oil/water-water/oil mixed phase), and also liposomes and transfersomes, glycerosomes, or plasters, ointments and creams, may be cited as examples for conventional application to the skin or mucosa.

Additional topically applicable forms that can be produced include pastes, foams, gels, powders, creams, ointments, and the like. As consistency-imparting bases, the pastes frequently comprise hydrophobic and hydrophilic auxiliary substances. In certain embodiments hydrophobic auxiliary substances are provide having a high solids content in order to increase their dispersity and flowability and glidability, and also to prevent agglomerates. In certain embodiments the powders or topically applicable powders can also contain, for example, starch types, such as wheat starch or rice starch, flame-disperse silicon dioxide or silicaceous earths, which also serve as diluents.

In addition to the known uses on the skin and/or mucosa, the following special preparations are preferably suitable for use as pharmaceuticals which can be administered topically, locally or regionally: emulsions, creams, ointments, foam tablets or suppositories which can be applied genitally, vaginally or rectally, in particular genitally and vaginally. Rectal capsules can also be produced on the basis of gelatin or other carrier substances. Examples of suitable suppository bases are hydrogenated fats, such as Witepsol®, Massa Estarium®, Novata®, coconut butter, glycerol/gelatin compositions, glycerol/saponaceous gels and polyethylene glycols.

The medicinal forms which are in each case suitable can be produced in conformity with the formulation specifications and procedures, based on pharmaceutical/physical principles, which are known to the skilled person.

Examples of suitable auxiliary substances and/or carrier substances are sodium alginate, as a gelatinizing agent for producing a suitable base, or cellulose derivatives, such as guar gum or xanthan gum, inorganic gelatinizing agents, such as aluminum hydroxides or bentonites (what are termed thixotropic gelatinizing agents), polyacrylic acid derivatives, such as Carbopol®, polyvinylpyrrolidone, microcrystalline cellulose or carboxymethylcellulose. In addition, amphiphilic low molecular weight and higher molecular weight compounds, and also phospholipids, are suitable. The gels can be present either as water-based hydrogels or as hydrophobic organogels, for example based on mixtures of low molecular weight and high molecular weight paraffin hydrocarbons and vaseline. The hydrophilic organogels can, for example, be prepared on the basis of high molecular weight polyethylene glycol. These gelatinous forms can be washed off. However, the organogels which are preferred are the hydrophobic organogels. Particular preference is given to hydrophobic auxiliary substances and additives such as petroleum jelly, wax, oleyl alcohol, propylene glycol monostearate and propylene glycol monopalmitostearate. Furthermore, it is possible to add dyes, for example yellow and/or red iron oxide and/or titanium dioxide, for the purpose of color adjustment.

Although not generally necessary, in certain embodiments, emulsifying agents can be included in the formulations. Examples of emulsifying agents that can be used include anionic, cationic or neutral surfactants, for example alkali soaps, metal soaps, amine soaps, sulfurized and sulfonated compounds, invert soaps, high fatty alcohols, partial fatty acid esters of sorbitan and polyoxyethylene sorbitan, e.g. lanette types, wool fat, lanolin and other synthetic products for producing oil/water and/or water/oil emulsions. Other examples of suitable auxiliary substances are ionic or anionic detergents, such as Triton X-100, Tween, sodium deoxycholate, and also polyols, such as polyethylene glycol or glycerol, sugars, for example sucrose or glucose, lipopolysaccharides, zwitterionic compounds, such as amino acids, such as glycine or, in particular, taurine or betaine, or lipids.

Vaseline, natural or synthetic waxes, fatty acids, fatty alcohols, fatty acid esters, for example as monoglycerides, diglycerides or triglycerides, paraffin or vegetable oils, hydrogenated castor oil or coconut oil, lard, synthetic fats, for example based on caprylic acid, capric acid, lauric acid and stearic acid, such as Softisan®, or triglyceride mixtures, such as Miglyol®, can be used as lipids in the form of fatty and/or oily and/or waxy components for producing the ointments, creams or emulsions.

In order to adjust the pH of the formulation, it is possible, for example, to use suitable organic or inorganic buffers, osmotically active acids and lyes, for example hydrochloric acid, citric acid, sodium hydroxide solution, potassium hydroxide solution or sodium hydrogen carbonate, and, in addition, buffer systems, such as citrate, phosphate buffer, Tris buffer (tris(hydroxymethyl)aminomethane, HEPES buffer ([4-(2-hydroxyethyl)piperazino]ethanesulfonic acid), MOPS buffer (3-morpholino-1-propanesulfonic acid) or triethanolamine. In general, the choice of the buffer depends on the buffer molarity that is desired.

In order to increase the stability, it is furthermore possible to add preservatives, such as methyl benzoate or propyl benzoate (parabene), sorbic acid, proteins, for example bovine, human or synthetic serum albumin, and/or protease inhibitors, such as aprotinin, .epsilon.-aminocarpoic acid, pepstatin A, EDTA or EGTA.

Auxiliary substances can also include penetration amplifiers, for example hydrophobic esters, such as isopropyl laureate, isopropyl myristate, isopropyl palmitate, isopropyl stearate, ethyl myristate, propyl myristate, butyl myristate and/or. ethyl oleate, in particular isopropyl myristate. In this connection, the term “hydrophobic” is understood as referring to compounds whose solubility in water is at most approx. 0.2 mg/ml, in particular at most approx. 0.1 mg/ml.

### Administration.

In various embodiments the topical virucidal compositions described herein can be administered singly or as multiple dosages throughout the day. For the control of HSV-1 infections, application of a virucidally effective amount of a composition according to the invention to an infected area, e.g., skin surfaces such as the area around the mouth, lips, mucous membranes, eyes, of an animal or human subject suffering from a viral infection, especially a herpes infection, will generally range from about one to three applications per day, for example at 20 minute intervals, depending upon the area to be treated, the severity of the symptoms and the nature of the virucidal agent and the topical vehicle employed.

For the control of genital herpes, i.e., HSV-2 infections, the compositions of this invention are administered to the site of infection (e.g., intravaginally) preferably in admixture with a pharmaceutical carrier. The carrier is, of course, chosen with regard to the intended route and method of administration. As described herein administration is typically topical, i.e., to a skin surface in a definite place or locus, for example, the vagina, the penis, etc., in the form of a cream, ointment, foam, jelly, gel, ovule or other suitable composition which lends itself to a topical dosage form. In certain embodiments Creams, gels, and ointments are preferred forms.

In certain embodiments the composition is preferably applied locally in the region in which there is a skin or mucosal change and/or disease.

The virucidal compositions described herein may also be used to prevent the spread of infection by viruses that reside in and/or are transmitted by and/or infect the cells of the dermis or epidermis. That is, in another embodiment the compositions described herein may be incorporated into a hand cream or lotion for use by medical personnel both before and after the examination of patients with suspected virus infections. The compositions described herein may be used in fluids used to kill virus on examining tables, instruments, gloves, towels and other surfaces which might come in contact with virus particles during the course of medical examinations. Further, all of the ingredients employed in the compositions described herein are generally recognized as safe. The low toxicity of the compositions described herein further enhances their attractiveness for such prophylactic use.

### EXAMPLES

The following examples are offered to illustrate, but not to limit the claimed invention.

### Example 1

The compositions are evaluated for the treatment of recurrent oral-facial Herpes simplex infections. A group of patients applies a solution comprising one of the formulations shown below in Table 1.

TABLE 1
Illustrative formulations.

| | Composition | | |
|---|---|---|---|
| Formulation | Components | Amount | Range |
| Formulation 1 | Salicylic acid | 1% | 0.1%-15% |
| | Ethanol | 10% | 10%-40% |
| | Remainder water | | |
| | pH | <4.5 | |
| Formulation 2 | Lactic acid | 0.7% | 0.6%-1.2% |
| | Ethanol | 10% | |
| | Remainder Water | | |
| | pH | <4.5 | |
| Formulation 3 | Coumaric acid | 1% | 0.5%-1.5% |
| | Ethanol | 10% | |
| | Remainder Water | | |
| | PH | <4.5 | |
| Formulation 4 | Mandelic acid | 1.2% | 0.7%-1.7% |
| | Ethanol | 10% | |
| | Remainder Water | | |
| | pH | <4.5 | |
| Formulation 5 | Ferulic Acid | 0.5% | 0.4-1.5% |
| | Vitamin C (ascorbic acid) | 15% | 5%-20% |
| | Ethanol | 10% | |
| | Remainder Water | | |
| | pH | <4.5 | |

Development of blisters is arrested and rapid crusting of the vesicles occurs within 2 to 3 days of treatment, as compared to 10 or more days without treatment. When the same composition is applied within 24 hours of the prodromal stage of infection, that is, during awareness of burning, tingling, or itching but before blister development, the a papule is not expected to occur. Thus, the compositions described herein are expected prevent the formulation of lesions, as well as being effective in reducing the healing time of the lesions.

### Example 2

### Efficacy of Topical Compounds in an Orofacial HSV-1 Infection in SKH-1 Mice

### Objective

The objective of this study was to evaluate protection from infection and/or disease provided by treatment with antiviral compounds against wild-type HSV-1 in a SKH-1 mouse model of Herpes labialis. The effect of treatment on viral shedding was also assessed. The two compounds were evaluated separately and as the composite compound.

### Description of the Model

It has been estimated that there are over 40 million people in the U.S. with Herpes labialis and there is a great need for the development of new antiviral drugs for this disease. We have utilized the SKH-1 strain of immunocompetent hairless mice to facilitate scoring of cutaneous lesions. Thus, orofacial inoculation of HSV-1 in these mice provides an appropriate model for testing new antiviral therapies.

In this model, mice are anesthetized with a ketamine/xylazine mixture and implanted with an electronic microchip for individual identification. Prior to inoculation, the snout, composed of the triangular shaped area over the nasal bones from the nose bridge to the eyes, is lightly abraded with a #113 tungsten-carbide engraving bit Dremmel tool. This procedure is performed carefully to prevent bleeding. This area is then swabbed for 10 seconds with a dacron swab soaked with HSV-1, E-377 at approximately 2×10⁶ pfu/ml. Following this procedure, animals are returned to their cages and observed until recovery.

Animals infected with HSV-1 in the orofacial area exhibit lesions that begin to appear on days 4-6 and are usually cleared by day 15. To determine the effect of treatment on cutaneous viral replication, severity of lesions is scored from days 1-21 and swabs of the snout area are taken on days 2-8 and 10. The samples are placed in 2.0 mls of media and frozen at −70° C. until titrated for HSV-1 on rabbit kidney fibroblast cells in a CPE microtiter plate assay. All drug studies are placebo or vehicle controlled.

### Experimental Design of Efficacy Study

Groups of SKH-1 females, approximately 5-6 weeks old at study initiation will be randomly assigned to five groups for efficacy (see Table 2.)

TABLE 2
Group assignments.

| Group | Treatment |
|---|---|
| 1 | Vehicle |
| 2 | Ethyl alcohol (CAS Number 64-17-5) diluted to 10% |
| | ethanol/distilled water |
| 3 | Glycolic acid (CAS Number 79-14-1) crystals are dissolved in |
| | distilled water with continual stirring until the solution reaches a |
| | pH of 2.45. |
| 4 | Composite Drug |
| | (Glycolic Acid [CAS Number 79-14-1] crystals are dissolved in |
| | 10% ethanol [see Compound 1 above] with continual stirring |
| | until the solution reaches |
| | a pH of 2.45.) |
| 5 | 5% Acyclovir in PEG |

The animals were inoculated orofacially with HSV-1, strain E-377, treated for 10 seconds, three times daily, 20 minutes apart, for three consecutive days beginning after 20% of infected mice developed lesions and observed for 21 days. Ethyl alcohol, glycolic acid and the combination of glycolic acid in 10% ethyl alcohol was prepared one day prior to use. During the post-challenge phase, animals were examined daily and lesions scored for severity and samples taken for quantitation of orofacial HSV replication.

### Methods

Viral Inoculation

Female SKH-1 mice (Charles River Laboratories, Raleigh, N.C.) were assigned randomly to groups. Each efficacy group contained 10 animals. Animals were anesthetized with ketamine-xylazine, implanted with an identification microchip, and has the orofacial area abraded using a Dremmel tool with an engraving bit. Viral inoculation was accomplished by rubbing a dacron-tipped swab soaked with HSV-1, strain E-377 at approximately 2×10⁶ pfu/ml (approximately 0.2 ml of virus dilution per swab) onto the abraded orofacial area for approximately 10 seconds.

Sample Collection, Virus Assays, Antibody Assays, and Scoring of Lesions

To determine the effect of treatment on HSV-1 replication on the orofacial area, swabs of the area were obtained on days 2 through 8 and 10 post-viral challenge. On treatment days, swabs were obtained at least 3 hours following the final treatment. Swabs were placed in tubes containing 2.0 ml of media, vortexed, and frozen at −70° C. until titrated for HSV. When all samples were collected they were thawed, diluted serially 10-fold with an autodilutor machine, and HSV-1 titers determined in rabbit kidney cells using a microtiter CPE assay. Wells were seeded with 10⁴ cells per well and inoculated in triplicate 24 h later. The plates were read under a microscope five days after viral inoculation and the presence/absence of viral CPE in each well noted. A TCID₅₀ value was calculated using Reed-Muench Methodology.

To determine the effect of treatment on HSV-1 replication from the lesions on the orofacial area, swabs of the area were obtained at least 3 hours after treatment on days when mice are treated. Thus, each animal had morning and afternoon swab samples from 3 consecutive days when mice were treated. Samples were taken before treatments and after treatments.

To determine the effect of treatment on the development and severity of lesions, severity of lesions were graded on a 0.0 to 5.0 scale in 0.5 increments, according to the scale below. The presence or absence of lesions and severity of lesions was recorded daily beginning day 1 through day 21 post-challenge. Animals were observed daily for signs of systemic viral disease.

Scale for Lesions on Face

0.0=normal

0.5 whole face red & swollen, 1-2 discrete lesions on face

1.0=3-5 discrete lesions on face

1.5≤25% confluency or scabs on face

2.0≤50% confluency or scabs on face

2.5≤75% confluency or scabs on face

3.0≤75-100% confluency or scabs on face

3.5=≤25% necrotic tissue

4.0=≤50% necrotic tissue

4.5=≤75% necrotic tissue

5.0=≤75-100% necrotic tissue

Statistical Evaluation

Mortality rates were analyzed by Fisher's exact test and mean day of death using Mann-Whitney U rank sum test. Infection rates, peak lesion scores, peak virus titers, areas under the curve for virus titer-day and lesion score-day between control and treated animals will be compared using the Mann-Whitney U Rank Sum Test. A P-value of 0.05 or less will be considered significant.

In addition, the immediate post treatment effects on lesion derived virus titers was analyzed using the mixed linear model with a P-value of 0.05 or less considered as significant. This difference in virus recovered from animals before and after treatments for all treatment days was used to calculate significance.

### Results.

The mortality of SKH-1 mice infected orofacially with HSV-1 and subsequently treated with single or combination therapy was not significantly decreased when compared to vehicle treated groups (Table 3). Mice treated topically with acyclovir, the positive control drug, had significantly reduced mortality (P<0.001).

TABLE 3
Effect of Topical Treatment with Antiviral Compounds on the
Mortality of SKH-1 Mice Infected Orofacially with HSV-1.

| | Mortality | | | | |
|---|---|---|---|---|---|
| Treatment^{a} | Number | Percent | P-value | MDD^{b} | P-value |
| Vehicle | | | | | |
| Sterile Distilled Water | 8/10 | 80 | — | 6.9 | — |
| Compound 1 | | | | | |
| Ethyl Alcohol 10% | 8/10 | 80 | NS^{c} | 7.9 | NS |
| Compound 2 | | | | | |
| Glycolic Acid | 7/9^{d} | 78 | NS | 8.1 | NS |
| Composite 1 + 2 | 8/10 | 80 | NS | 7.4 | NS |
| Acyclovir 5% | 0/10 | 0 | <0.001 | — | — |

^{a}Topical treatments with vehicle, Compound 1, Coumpound 2, or Composite 1 + 2 were applied topically 3 times daily for 3 consecutive days (Days 4-6) in 20 min. intervals beginning when 20% mice displayed lesions. Acyclovir (5%) was treated 3 times daily for 5 days beginning 24 hours after virus inoculation as usual (~6 hours apart).
^{b}MDD = Mean Day of Death.
^{c}NS = Not significant when compared to the appropriate placebo control.
^{d}One mortality due to trauma on Day 5 of study.

The results from the lesion scores (Table 4) indicated that acyclovir treatment significantly reduced the lesion score area under the curve values and mean peak lesion scores (p<0.001) when compared to vehicle treated groups. Mean peak lesion scores were not significantly reduced by either single compound or combination treatments. On the contrary, the lesion score area under the curve value (P<0.01) and mean peak lesion scores (P=0.001) were significantly increased in mice treated with 10% ethyl alcohol alone.

TABLE 4
effect of topical treatment with antiviral compounds on the facial
lesion development of skh-1 mice infected orofacially with HSV-1.

| | #With | Lesion- | | Mean Peak | |
|---|---|---|---|---|---|
| | Lesions/ | Day | | Lesion | |
| Treatment^{a} | #Inoculated | AUC | P-Value | Score ± SD | P-Value |
| Vehicle^{b} | 10/10 | 21.4 | — | 2.7 ± 0.8 | — |
| Compound 1 | | | | | |
| Ethyl Alcohol 10% | 10/10 | 41.1↑ | <0.01 | 3.0 ± 0.0↑ | 0.001 |
| Compound 2 | | | | | |
| Glycolic Acid | 9/9^{d} | 29.2 | NS^{c} | 2.6 ± 0.96 | NS |
| Composite (1 + 2) | 9/10 | 18.4 | NS | 2.6 ± 0.96 | NS |
| 5% Acyclovir | 0/10 | 0.0 | <0.001 | 0.0 ± 0.0 | <0.001 |

^{a}Topical treatments with vehicle, Compound 1, Compound 2 , or Composite 1 + 2 were applied topically 3 times daily for 3 consecutive days (Days 4-6) in 20 min. intervals beginning when 20% mice displayed lesions. Acyclovir (5%) was treated 3 times daily for 5 days beginning 24 hours after virus inoculation as usual (~6 hours apart).
^{b}Sterile distilled water.
^{c}NS = non-significant when compared to the appropriate controls.
^{d}One mortality due to trauma on Day 5 of study.

The acyclovir treated group had significantly reduced virus titer-day AUC values and reduced mean peak virus titers (Table 5, P<0.001). The compounds administered individually or together as a combination were considered ineffective since there was no statistically significant reduction in viral replication over the course of the infection.

TABLE 5
Effect of topical treatment with antiviral compounds on virus
titers in an orofacial HSV-1 infection of SKH-1 mice

| | #Virus | | | | |
|---|---|---|---|---|---|
| | Positive/ | Titer- | | Mean Peak | |
| | #Inocu- | Day | | Titer | |
| Treatment^{a} | lated | AUC | P-Value | Score ± SD | P-Value |
| Vehicle^{b} | 10/10 | 27.6 | — | 6.0 ± 0.1 | — |
| Compound 1 | 10/10 | 32.2 | NS | 6.0 ± 0.2 | NS |
| Compound 2 | 10/10 | 33.3 | NS | 5.3 ± 0.5 | NS |
| Composite (1 + 2) | 10/10 | 30.0 | NS | 5.7 ± 0.1 | NS |
| 5% Acyclovir | 3/10 | 0.6 | <0.001 | 0.6 ± 0.1 | 0.001 |

^{a}Topical treatments with vehicle, Compound 1, Compound 2 , or Composite 1 + 2 were applied topically 3 times daily for 3 consecutive days (Days 4-6) in 20 min. intervals beginning when 20% mice displayed lesions. Acyclovir (5%) was treated 3 times daily for 5 days beginning 24 hours after virus inoculation as usual (~6 hours apart).
^{b}Sterile distilled water.
^{c}NS = non-significant when compared to the appropriate controls.

When evaluating effects on viral replication before and after treatment, the only significant effect was achieved by composite treatments (P=0.0447) as determined by the mixed linear model group statistical evaluation (Table 6).

TABLE 6
Before and After Treatment Effects of Antiviral Compounds on Virus Titers in an
Orofacial HSV-1 Infection of SKH-1 Mice.

| | Day 4 | | Day 5 | | Day 6 | | |
|---|---|---|---|---|---|---|---|
| Treatment^{a} | am | pm | am | pm | am | pm | p-value |
| Vehicle^{b} | 5.3 ± 1.5^{c} | 4.9 ± 1.9 | 5.0 ± 1.7 | 5.0 ± 1.5 | 4.1 ± 1.7 | 2.9 ± 1.4 | — |
| Compound 1 | 5.1 ± 1.0 | 5.1 ± 1.5 | 5.7 ± 0.4 | 5.6 ± 0.6 | 4.9 ± 0.7 | 4.0 ± 0.8 | NS^{d} |
| Compound 2 | 4.2 ± 1.3 | 4.2 ± 1.6 | 5.5 ± 0.4 | 5.1 ± 0.6 | 5.2 ± 0.6 | 4.4 ± 0.5 | NS |
| Composite (1 + 2) | 5.0 ± 1.6 | 4.4 ± 1.7 | 5.4 ± 0.9 | 4.5 ± 1.4 | 4.8 ± 1.5 | 3.5 ± 1.5 | 0.0447^{e} |
| 5% Acyclovir | 0.2 ± 0.8 | 0.0 ± 0.0 | 0.0 ± 0.0 | 0.0 ± 0.0 | 0.0 ± 0.0 | 0.0 ± 0.0 | NS |

^{a}Topical treatments with vehicle, Compound 1, Compound 2 or Composite 1 + 2 were applied topically 3 times daily for 3 consecutive days (Days 4-6) in 20 min. intervals beginning when 20% mice displayed lesions. Acyclovir (5%) was treated 3 times daily for 5 days beginning 24 hours after virus inoculation as usual (~6 hours apart).
^{b}Sterile distilled water.
^{c}Values represent mean virus titer as determined by Reed-Muench as TCID₅₀ ± standard deviation.
^{d}NS = non-significant when compared to the appropriate vehicle controls by mixed linear model statistical evaluation.

### CONCLUSIONS

The combination of glycolic acid with 10% ethyl alcohol appeared to have significant activity in the orofacial model against HSV-1 infection in SKH-1 mice, but only in the immediate post-treatment time period. Ethanol alone or glycolic acid alone had no significant effect on improving mortality, lesion development, reduction in virus titer over the entire experimental period, or in immediate post-treatment virus titers. Composite treatment had no significant effect on improving mortality, lesion development, or reduction in virus titer over the entire experimental period. Mice treated with ethanol alone even had some increase in lesion day area under the curve values and increased peak lesion scores. The activity of 5% acyclovir, the positive control drug, demonstrated highest efficacy with significant increases in survival, reduced lesion development, and reduced virus titers over the entire experimental treatment.

The differences between groups included % change from each of days 4, 5 and 6. Using mixed linear model (PROC MIXED in SAS), group 4 had more viral reduction (AM to PM) compared to group 1 (p=0.0447), while group 2 and group 3 did not have this effect (p=0.8843 and 0.9638, respectively).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition that inactivates or inhibits replication of a virus, said composition comprising:
a C₁-C₁₀ alcohol;
an acid selected from the group consisting of gallic acid, methyl gallate, 3,4-dihydroxy benzoic acid, p-hydroxy benzoic acid, vanillic acid, p-coumaric acid, ferulic acid, syringic acid, salicylic acid, luteic acid, monochloroacetic acid, squaric acid, tartaric acid, retinoic acid, and eudesmic acid; and
a pharmaceutically acceptable carrier;
where the pH of said composition is pH 4.5 or lower.

2. A composition that inactivates or inhibits replication of a virus, said composition comprising:
a C₁-C₁₀ alcohol;
a beta hydroxy acid or an omega hydroxy acid; and
a pharmaceutically acceptable carrier;
where the pH of said composition is pH 4.5 or lower.

3. The composition of claim 2, wherein said acid comprises a beta hydroxy acid.

4. The composition of claim 3, wherein said acid comprises a beta hydroxyl acid selected from the group consisting of salicylic acid, propanoic acid, α-hydroxypropinoic acid, and β-hydroxypropinoic acid.

5. The composition of claim 2, wherein said acid comprises an omega hydroxy acid.

6. The composition of claim 5, wherein said acid comprises an omega hydroxy acid selected from the group consisting of 16-hydroxy palmitic acid, 18-hydroxy stearic acid, ω-hydroxydotriacontanoic acid, ω-hydroxytetracosanoic acid, ω-hydroxytriacontanoic acid, E)-10-hydroxydec-2-enoic acid, 10-hydroxycapric acid, 12-hydroxylauric acid, 14-hydroxymyristic acid, 22-hydroxydocosanoic acid, 26-hydroxyhexacosanoic acid, 3,12-dihydroxylauric acid, 3,16-dihydroxypalmitic acid, 3,18-dihydroxystearic acid, 5-hydroxypentanoic acid, 6-hydroxyhexanoic acid, and juniperic acid.

7. The composition according to any one of claims 1-6, wherein said composition further comprises one or more agents selected from the group consisting of an antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide.

8. The composition of claim 7, wherein said composition comprises a long or short chain ceramide.

9. The composition of claim 7, wherein said composition comprises an allantoin.

10. The composition of claim 7, wherein said composition comprises urea.

11. The composition of claim 7, wherein said composition comprises a retinoid.

12. The composition of claim 7, wherein said composition comprises an antioxidant phenolic compound selected from the group consisting of phenol, and resorcinol.

13. The composition of claim 7, wherein said composition comprises a dicarboxylic acid selected from the group consisting of succinic acid, and tartaric acid.

14. The composition according to any one of claims 1-13, wherein said alcohol comprises one or more alcohols selected from the group consisting of methanol, ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol.

15. A composition that inactivates or inhibits replication of a virus, said composition comprising:
a C₁-C₁₀ alcohol;
an acid;
an additional compound selected from the group consisting of antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide; and
a pharmaceutically acceptable carrier;
where the pH of said composition is pH 4.5 or lower.

16. The composition of claim 15, wherein said composition comprises an antioxidant phenolic compound selected from the group consisting of phenol, and resorcinol.

17. The composition of claim 15, wherein said composition comprises a dicarboxylic acid selected from the group consisting of succinic acid, and tartaric acid.

18. The composition according to any one of claims 15-17, wherein said alcohol comprises one or more alcohols selected from the group consisting of ethanol, 2-propanol, 1-propanol, 2,3-butanediol, 1,2-butanediol, 1,3-butanediol, and 1,4-butanediol, butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol.

19. The composition according to any one of claims 15-18, wherein said acid comprises one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid.

20. A composition that inactivates or inhibits replication of a virus, said composition comprising:
one or more alcohols selected from the group consisting of butyl alcohol (including n-butanol, sec-butanol, isobutanol, tert-butanol), pentanol, hexadecan-1-ol, ethane-1,2-diol, propane-1,2-diol, propane-1,2,3-triol, butane-1,2,3,4-tetraol, pentane-1,2,3,4,5-pentol, hexane-1,2,3,4,5,6-hexol, heptane-1,2,3,4,5,6,7-heptol, prop-2-ene-1-ol, 3,7-dimethylocta-2,6-dien-1-ol, prop-2-in-1-ol, cyclohexane-1,2,3,4,5,6-hexol, and 2-(2-propyl)-5-methyl-cyclohexane-1-ol;
an acid; and
a pharmaceutically acceptable carrier;
where the pH of said composition is pH 4.5 or lower.

21. The composition of claim 20, wherein said composition further comprises one or more agents selected from the group consisting of an antioxidant phenolic compound, a tannin, a dicarboxylic acids or derivative thereof, mandelic acid, an extract from the plant Prunella vulgaris, vitamin C, urea, allantoin, an emollient, a retinoid, and a long or short chain ceramide.

22. The composition of claim 21, wherein said composition comprises an antioxidant phenolic compound selected from the group consisting of phenol, and resorcinol.

23. The composition of claim 21, wherein said composition comprises a dicarboxylic acid selected from the group consisting of succinic acid, and tartaric acid.

24. The composition according to any one of claims 20-23, wherein said acid comprises one or more acids selected from the group consisting of glycolic acid, lactic acid, succinic acid, malic acid, citric acid, acetic acid, formic acid, oxalic acid, uric acid, hydrochloric acid, nitric acid, phosphoric acid, sulphuric acid, boric acid, hydrobromic acid, hydroiodic acid, hydrosulfuric acid, perchloric acid, gallic acid, methyl gallate, 3,4-dihydroxy benzoic acid, p-hydroxy benzoic acid, vanillic acid, p-coumaric acid, ferulic acid, syringic acid, salicylic acid, luteic acid, and eudesmic acid.

25. The composition according to any one of claims 1-24, wherein the pH of said composition ranges from pH 2.0 to about pH 4.5.

26. The composition according to any one of claims 1-24, wherein the pH of said composition ranges is about pH 3.5 or lower.

27. The composition of claim 25, wherein the pH of said composition is about pH 3.0 or lower.

28. The composition of claim 25, wherein the pH of said composition is about pH 2.5 or lower.

29. The composition according to any one of claims 1-28, wherein said alcohol ranges from about 0.2% by volume up to about 40% by volume of said composition.

30. The composition according to any one of claims 1-28, wherein said alcohol ranges from about 0.5% by volume up to about 20% by volume of said composition.

31. The composition according to any one of claims 1-28, wherein said alcohol ranges from about 1% by volume up to about 15% by volume of said composition.

32. The composition according to any one of claims 1-31, wherein said composition excludes an amphoteric or pseudoamphoteric compound.

33. The composition of claim 32, wherein said composition excludes an amino acid and/or a peptide.

34. The composition according to any one of claims 32-33, wherein said composition excludes a dipeptide and/or a tripeptide.

35. The composition according to any one of claims 32-34, wherein said composition excludes an imidazoline amphoteric and/or a lecithin amphoteric.

36. The composition of claim 35, wherein said composition excludes one or more compounds selected from the group consisting of cocoamphoglycine, cocoamphoproprionate, cocoamphopropylsulfonate, phosphatidyl ethanolamine, phosphatidyl serine, and sphingomyelin.

37. The composition according to any one of claims 1-36, wherein said composition is formulated as a unit dosage formulation.

38. The composition according to any one of claims 1-37, wherein said composition is formulated as a preparation selected from the group consisting of a fluid, roll-on, spray, tincture, gel, ointment, cream, salve, lotion, lip balm, foam, spray, and aerosol.

39. A method of inactivating or inhibiting replication of a virus in a mammal, said method comprising:
administering to said mammal a composition according to any one of claims 1-38 in an amount sufficient to inhibit, inactivate or kill said virus.

40. The method of claim 39, wherein said administering reduces or inhibits viral-induced lesions in said mammal when said mammal is infected by said virus.

41. The method of claim 39, wherein said virus resides in the dermis or epidermis of a human or animal infected by said virus.

42. The method according to any one of claims 39-41, wherein said composition is applied topically to said mammal.

43. The method of claim 42, wherein said composition is applied to lesions on said mammal.

44. The method according to any one of claims 39-43, wherein said mammal is a human.

45. The method according to any one of claims 39-44, wherein said mammal is infected with said virus or at risk for an infection by said virus.

46. The method of claim 45, wherein said mammal is infected with said virus.

47. The method according to any one of claims 39-46, wherein said virus is a member of the Herpesviridae family.

48. The method of claim 47, wherein said virus is a virus selected from the group consisting of Herpes simplex virus-1 (HSV-1), Herpes simplex virus-2 (HSV-2), Varicella zoster virus (VZV), Epstein-Barr virus (EBV), lymphocryptovirus, Cytomegalovirus (CMV), Roseolovirus, Herpes lymphotropic virus, Pityriasis rosea, and Kaposi's sarcoma-associated herpesvirus (KSHV).

49. The method of claim 47, wherein said virus is Herpes simplex 1.

50. The method of claim 47, wherein said virus is Herpes simplex 2.

51. The method of claim 47, wherein said virus is Varicella zoster virus.

52. The method according to any one of claims 39-42, wherein said virus is a member of the Poxviridae family.

53. The method of claim 52, wherein said virus is a virus selected from the group consisting of smallpox virus (variola), vaccinia virus, cowpox virus, monkeypox virus, orf virus, pseudocowpox, bovine papular stomatitis virus, tanapox virus, Molluscum contagiosum virus (MCV). wherein said mammal is infected with said virus or at risk for an infection by said virus.

54. The method of claim 52, wherein said virus is Molluscum contagiosum.

55. The method according to any one of claims 39-42, wherein said virus is selected from the group consisting of rhinoviruses, adenoviruses, enteroviruses, cornoviruses, respiratory syncytial viruses, influenza viruses and parainfluenza viruses.
